SEQUENCE LISTING (114 sequences)

SEQ ID NO: 1 - DNA, 35 bp, synthetic construct
  misc_feature 1..35: note = PCR primer - PCR1
  source 1..35: mol_type = other DNA
gtctcgtgggctcgggatctcttcagcgtgggagg

SEQ ID NO: 2 - DNA, 35 bp, synthetic construct
  misc_feature 1..35: note = PCR primer - PCR1
  source 1..35: mol_type = other DNA
gtctcgtgggctcggctgagagggtgaggacagga

SEQ ID NO: 3 - DNA, 35 bp, synthetic construct
  misc_feature 1..35: note = PCR primer - PCR1
  source 1..35: mol_type = other DNA
gtctcgtgggctcgggagagggaggaacgcatagc

SEQ ID NO: 4 - DNA, 35 bp, synthetic construct
  misc_feature 1..35: note = PCR primer - PCR1
  source 1..35: mol_type = other DNA
gtctcgtgggctcggagggagcagaattgaggcac

SEQ ID NO: 5 - DNA, 35 bp, synthetic construct
  misc_feature 1..35: note = PCR primer - PCR1
  source 1..35: mol_type = other DNA
gtctcgtgggctcgggtccccaggtaacatccacg

SEQ ID NO: 6 - DNA, 35 bp, synthetic construct
  misc_feature 1..35: note = PCR primer - PCR1
  source 1..35: mol_type = other DNA
gtctcgtgggctcggagctataagccaagggacgc

SEQ ID NO: 7 - DNA, 35 bp, synthetic construct
  misc_feature 1..35: note = PCR primer - PCR1
  source 1..35: mol_type = other DNA
gtctcgtgggctcgggtctagagtgggaggggcta

SEQ ID NO: 8 - DNA, 36 bp, synthetic construct
  misc_feature 1..36: note = PCR primer - PCR1
  source 1..36: mol_type = other DNA
gtctcgtgggctcggccagctggctgaaatgaagaa

SEQ ID NO: 9 - DNA, 35 bp, synthetic construct
  misc_feature 1..35: note = PCR primer - PCR1
  source 1..35: mol_type = other DNA
gtctcgtgggctcggtagttgaggggtggctttgc

SEQ ID NO: 10 - DNA, 35 bp, synthetic construct
  misc_feature 1..35: note = PCR primer - PCR1
  source 1..35: mol_type = other DNA
gtctcgtgggctcggcctacctccctgttctctgc

SEQ ID NO: 11 - DNA, 35 bp, synthetic construct
  misc_feature 1..35: note = PCR primer - PCR1
  source 1..35: mol_type = other DNA
gtctcgtgggctcgggtgggcagcttccagtagtt

SEQ ID NO: 12 - DNA, 35 bp, synthetic construct
  misc_feature 1..35: note = PCR primer - PCR1
  source 1..35: mol_type = other DNA
gtctcgtgggctcggctgagactcggggtttgcat

SEQ ID NO: 13 - DNA, 54 bp, synthetic construct
  misc_feature 1..54: note = ont-BC01
  source 1..54: mol_type = other DNA
gtctcgtccgctcggcacaaagacaccgacaactttcttgtctcgtgggctcgg

SEQ ID NO: 14 - DNA, 54 bp, synthetic construct
  misc_feature 1..54: note = ont-BC02
  source 1..54: mol_type = other DNA
gttagttgatgtagtacagacgactacaaacggaatcgagtctcgtgggctcgg

SEQ ID NO: 15 - DNA, 54 bp, synthetic construct
  misc_feature 1..54: note = ont-BC03
  source 1..54: mol_type = other DNA
gtctcgtccgctcggcctggtaactgggacacaagactcgtctcgtgggctcgg

SEQ ID NO: 16 - DNA, 54 bp, synthetic construct
  misc_feature 1..54: note = ont-BC04
  source 1..54: mol_type = other DNA
gttagttgatgtagttagggaaacacgatagaatccgaagtctcgtgggctcgg

SEQ ID NO: 17 - DNA, 54 bp, synthetic construct
  misc_feature 1..54: note = ont-BC05
  source 1..54: mol_type = other DNA
gtctcgtccgctcggaaggttacacaaaccctggacaaggtctcgtgggctcgg

SEQ ID NO: 18 - DNA, 54 bp, synthetic construct
  misc_feature 1..54: note = ont-BC06
  source 1..54: mol_type = other DNA
gttagttgatgtagtgactactttctgcctttgcgagaagtctcgtgggctcgg

SEQ ID NO: 19 - DNA, 54 bp, synthetic construct
  misc_feature 1..54: note = ont-BC07
  source 1..54: mol_type = other DNA
gatatgatatagataaaggattcattcccacggtaacacgtctcgtgggctcgg

SEQ ID NO: 20 - DNA, 54 bp, synthetic construct
  misc_feature 1..54: note = ont-BC08
  source 1..54: mol_type = other DNA
gttagttgatgtagtacgtaacttggtttgttccctgaagtctcgtgggctcgg

SEQ ID NO: 21 - DNA, 54 bp, synthetic construct
  misc_feature 1..54: note = ont-BC09
  source 1..54: mol_type = other DNA
gtctcgtccgctcggaaccaagactcgctgtgcctagttgtctcgtgggctcgg

SEQ ID NO: 22 - DNA, 54 bp, synthetic construct
  misc_feature 1..54: note = ont-BC10
  source 1..54: mol_type = other DNA
gttagttgatgtagtgagaggacaaaggtttcaacgcttgtctcgtgggctcgg

SEQ ID NO: 23 - DNA, 54 bp, synthetic construct
  misc_feature 1..54: note = ont-BC11
  source 1..54: mol_type = other DNA
gttagttgatgtagttccattccctccgatagatgaaacgtctcgtgggctcgg

SEQ ID NO: 24 - DNA, 54 bp, synthetic construct
  misc_feature 1..54: note = ont-BC12
  source 1..54: mol_type = other DNA
gtctcgtccgctcggtccgattctgcttctttctacctggtctcgtgggctcgg

SEQ ID NO: 25 - DNA, 54 bp, synthetic construct
  misc_feature 1..54: note = ont-BC13
  source 1..54: mol_type = other DNA
gtctcgtccgctcggtcacacgagtatggaagtcgttctgtctcgtgggctcgg

SEQ ID NO: 26 - DNA, 54 bp, synthetic construct
  misc_feature 1..54: note = ont-BC14
  source 1..54: mol_type = other DNA
tacattgatgcatggtctatgggtcccaagagactcgttgtctcgtgggctcgg

SEQ ID NO: 27 - DNA, 54 bp, synthetic construct
  misc_feature 1..54: note = ont-BC15
  source 1..54: mol_type = other DNA
gttagttgatgtagtcagtggtgttagcgaggtagacctgtctcgtgggctcgg

SEQ ID NO: 28 - DNA, 54 bp, synthetic construct
  misc_feature 1..54: note = ont-BC16
  source 1..54: mol_type = other DNA
tacattgatgcatggagtacgaaccactgtcagttgacggtctcgtgggctcgg

SEQ ID NO: 29 - DNA, 54 bp, synthetic construct
  misc_feature 1..54: note = ont-BC17
  source 1..54: mol_type = other DNA
gtctcgtccgctcggatcagaggtactttcctggagggtgtctcgtgggctcgg

SEQ ID NO: 30 - DNA, 54 bp, synthetic construct
  misc_feature 1..54: note = ont-BC18
  source 1..54: mol_type = other DNA
gttagttgatgtagtgcctatctaggttgttgggtttgggtctcgtgggctcgg

SEQ ID NO: 31 - DNA, 54 bp, synthetic construct
  misc_feature 1..54: note = ont-BC19
  source 1..54: mol_type = other DNA
gttagttgatgtagtatctcttgacactgcacgaggaacgtctcgtgggctcgg

SEQ ID NO: 32 - DNA, 54 bp, synthetic construct
  misc_feature 1..54: note = ont-BC20
  source 1..54: mol_type = other DNA
gttagttgatgtagtatgagttctcgtaacaggacgcaagtctcgtgggctcgg

SEQ ID NO: 33 - DNA, 54 bp, synthetic construct
  misc_feature 1..54: note = ont-BC21
  source 1..54: mol_type = other DNA
gttagttgatgtagttagagaacggacaatgagaggctcgtctcgtgggctcgg

SEQ ID NO: 34 - DNA, 54 bp, synthetic construct
  misc_feature 1..54: note = ont-BC22
  source 1..54: mol_type = other DNA
gttagttgatgtagtcgtactttgatacatggcagtggtgtctcgtgggctcgg

SEQ ID NO: 35 - DNA, 54 bp, synthetic construct
  misc_feature 1..54: note = ont-BC23
  source 1..54: mol_type = other DNA
gtctcgtccgctcggcgaggaggttcactgggtagtaaggtctcgtgggctcgg

SEQ ID NO: 36 - DNA, 54 bp, synthetic construct
  misc_feature 1..54: note = ont-BC24
  source 1..54: mol_type = other DNA
gttagttgatgtagtctaacccatcatgcagaactatgcgtctcgtgggctcgg

SEQ ID NO: 37 - DNA, 54 bp, synthetic construct
  misc_feature 1..54: note = ont-BC25
  source 1..54: mol_type = other DNA
gtctcgtccgctcggcattgcgttgcatacccaacttacgtctcgtgggctcgg

SEQ ID NO: 38 - DNA, 54 bp, synthetic construct
  misc_feature 1..54: note = ont-BC26
  source 1..54: mol_type = other DNA
tacattgatgcatggatgagaatgcgtagtcgctgtatggtctcgtgggctcgg

SEQ ID NO: 39 - DNA, 54 bp, synthetic construct
  misc_feature 1..54: note = ont-BC27
  source 1..54: mol_type = other DNA
gtctcgtccgctcggtgtaagaggtgaatctaaccgtcggtctcgtgggctcgg

SEQ ID NO: 40 - DNA, 54 bp, synthetic construct
  misc_feature 1..54: note = ont-BC28
  source 1..54: mol_type = other DNA
gttagttgatgtagtgatacggtgccttcttaggtttcagtctcgtgggctcgg

SEQ ID NO: 41 - DNA, 54 bp, synthetic construct
  misc_feature 1..54: note = ont-BC29
  source 1..54: mol_type = other DNA
gttagttgatgtagtggtctgtcaacccaaggtgtctaggtctcgtgggctcgg

SEQ ID NO: 42 - DNA, 54 bp, synthetic construct
  misc_feature 1..54: note = ont-BC30
  source 1..54: mol_type = other DNA
gttagttgatgtagttgggtcgaagtagatcctcactgagtctcgtgggctcgg

SEQ ID NO: 43 - DNA, 54 bp, synthetic construct
  misc_feature 1..54: note = ont-BC31
  source 1..54: mol_type = other DNA
gtctcgtccgctcggcaatgtaactgattgctgtacgcagtctcgtgggctcgg

SEQ ID NO: 44 - DNA, 54 bp, synthetic construct
  misc_feature 1..54: note = ont-BC32
  source 1..54: mol_type = other DNA
gttagttgatgtagtatgacgttgtcggacttctactgggtctcgtgggctcgg

SEQ ID NO: 45 - DNA, 54 bp, synthetic construct
  misc_feature 1..54: note = ont-BC33
  source 1..54: mol_type = other DNA
gtctcgtccgctcggagttacccaaccgtaccaagtctggtctcgtgggctcgg

SEQ ID NO: 46 - DNA, 54 bp, synthetic construct
  misc_feature 1..54: note = ont-BC34
  source 1..54: mol_type = other DNA
gttagttgatgtagtgcctttgacttgagttcttcgtccgtctcgtgggctcgg

SEQ ID NO: 47 - DNA, 54 bp, synthetic construct
  misc_feature 1..54: note = ont-BC35
  source 1..54: mol_type = other DNA
gtctcgtccgctcgggcagtccctcagcttcgtaagtaggtctcgtgggctcgg

SEQ ID NO: 48 - DNA, 54 bp, synthetic construct
  misc_feature 1..54: note = ont-BC36
  source 1..54: mol_type = other DNA
gttagttgatgtagttgtttcctcctctaactgggacatgtctcgtgggctcgg

SEQ ID NO: 49 - DNA, 54 bp, synthetic construct
  misc_feature 1..54: note = ont-BC37
  source 1..54: mol_type = other DNA
gtctcgtccgctcggtgatactaagcatcaatcgcaagcgtctcgtgggctcgg

SEQ ID NO: 50 - DNA, 54 bp, synthetic construct
  misc_feature 1..54: note = ont-BC38
  source 1..54: mol_type = other DNA
gttagttgatgtagtttctctgtatcgtcctcctgtggtgtctcgtgggctcgg

SEQ ID NO: 51 - DNA, 54 bp, synthetic construct
  misc_feature 1..54: note = ont-BC39
  source 1..54: mol_type = other DNA
gttagttgatgtagtgagaggctctagttgacactgtgggtctcgtgggctcgg

SEQ ID NO: 52 - DNA, 54 bp, synthetic construct
  misc_feature 1..54: note = ont-BC40
  source 1..54: mol_type = other DNA
gttagttgatgtagtggctatccttggtcatccaaactagtctcgtgggctcgg

SEQ ID NO: 53 - DNA, 54 bp, synthetic construct
  misc_feature 1..54: note = ont-BC41
  source 1..54: mol_type = other DNA
gttagttgatgtagtcgtgtacttctctggacgaactccgtctcgtgggctcgg

SEQ ID NO: 54 - DNA, 54 bp, synthetic construct
  misc_feature 1..54: note = ont-BC42
  source 1..54: mol_type = other DNA
gtctcgtccgctcggctggcaggtatgccttacacgtaggtctcgtgggctcgg

SEQ ID NO: 55 - DNA, 54 bp, synthetic construct
  misc_feature 1..54: note = ont-BC43
  source 1..54: mol_type = other DNA
gttagttgatgtagtctaccgtcgagtcaacaacgaaaggtctcgtgggctcgg

SEQ ID NO: 56 - DNA, 54 bp, synthetic construct
  misc_feature 1..54: note = ont-BC44
  source 1..54: mol_type = other DNA
gttagttgatgtagtgagtgggaaggaaccctttctactgtctcgtgggctcgg

SEQ ID NO: 57 - DNA, 54 bp, synthetic construct
  misc_feature 1..54: note = ont-BC45
  source 1..54: mol_type = other DNA
gtctcgtccgctcggcactgaaggcatctctgttggatcgtctcgtgggctcgg

SEQ ID NO: 58 - DNA, 54 bp, synthetic construct
  misc_feature 1..54: note = ont-BC46
  source 1..54: mol_type = other DNA
gttagttgatgtagtcaggagaatgaagtggaacacagcgtctcgtgggctcgg

SEQ ID NO: 59 - DNA, 54 bp, synthetic construct
  misc_feature 1..54: note = ont-BC47
  source 1..54: mol_type = other DNA
gtctcgtccgctcgggaactacctgtgggaaagttgcacgtctcgtgggctcgg

SEQ ID NO: 60 - DNA, 54 bp, synthetic construct
  misc_feature 1..54: note = ont-BC48
  source 1..54: mol_type = other DNA
gttagttgatgtagttacaggtgtaccacgttccagatggtctcgtgggctcgg

SEQ ID NO: 61 - DNA, 54 bp, synthetic construct
  misc_feature 1..54: note = ont-BC49
  source 1..54: mol_type = other DNA
gtctcgtccgctcggctagatgttcaaagctgcaccagtgtctcgtgggctcgg

SEQ ID NO: 62 - DNA, 54 bp, synthetic construct
  misc_feature 1..54: note = ont-BC50
  source 1..54: mol_type = other DNA
gttagttgatgtagtacgcaggaagttaccaaagtccatgtctcgtgggctcgg

SEQ ID NO: 63 - DNA, 54 bp, synthetic construct
  misc_feature 1..54: note = ont-BC51
  source 1..54: mol_type = other DNA
gtctcgtccgctcgggaggacccagtaggctcattcaacgtctcgtgggctcgg

SEQ ID NO: 64 - DNA, 54 bp, synthetic construct
  misc_feature 1..54: note = ont-BC52
  source 1..54: mol_type = other DNA
tacattgatgcatgggtccacgaacaatcttgtctctcagtctcgtgggctcgg

SEQ ID NO: 65 - DNA, 54 bp, synthetic construct
  misc_feature 1..54: note = ont-BC53
  source 1..54: mol_type = other DNA
gtctcgtccgctcggctttgcatgagacggtctgaatctgtctcgtgggctcgg

SEQ ID NO: 66 - DNA, 54 bp, synthetic construct
  misc_feature 1..54: note = ont-BC54
  source 1..54: mol_type = other DNA
gttagttgatgtagtcatgctccttagtcaaagctcttggtctcgtgggctcgg

SEQ ID NO: 67 - DNA, 54 bp, synthetic construct
  misc_feature 1..54: note = ont-BC55
  source 1..54: mol_type = other DNA
gtctcgtccgctcggcgtagatcagggtctcatcttccagtctcgtgggctcgg

SEQ ID NO: 68 - DNA, 54 bp, synthetic construct
  misc_feature 1..54: note = ont-BC56
  source 1..54: mol_type = other DNA
gtctcgtccgctcggttcatgccacctgttgagtagtgagtctcgtgggctcgg

SEQ ID NO: 69 - DNA, 54 bp, synthetic construct
  misc_feature 1..54: note = ont-BC57
  source 1..54: mol_type = other DNA
tacattgatgcatggacttccgaaggagattgacctagcgtctcgtgggctcgg

SEQ ID NO: 70 - DNA, 54 bp, synthetic construct
  misc_feature 1..54: note = ont-BC58
  source 1..54: mol_type = other DNA
gttagttgatgtagttcagactcacggaggagtaacctggtctcgtgggctcgg

SEQ ID NO: 71 - DNA, 54 bp, synthetic construct
  misc_feature 1..54: note = ont-BC59
  source 1..54: mol_type = other DNA
gttagttgatgtagtaccttgctttcccttcttgattgagtctcgtgggctcgg

SEQ ID NO: 72 - DNA, 54 bp, synthetic construct
  misc_feature 1..54: note = ont-BC60
  source 1..54: mol_type = other DNA
gttagttgatgtagtccatagaagccttggttgaacatggtctcgtgggctcgg

SEQ ID NO: 73 - DNA, 54 bp, synthetic construct
  misc_feature 1..54: note = ont-BC61
  source 1..54: mol_type = other DNA
tacattgatgcatgggtgctgaggcacatagtaccctctgtctcgtgggctcgg

SEQ ID NO: 74 - DNA, 54 bp, synthetic construct
  misc_feature 1..54: note = ont-BC62
  source 1..54: mol_type = other DNA
gttagttgatgtagttacgtcctgaagtaagtgtgggtggtctcgtgggctcgg

SEQ ID NO: 75 - DNA, 54 bp, synthetic construct
  misc_feature 1..54: note = ont-BC63
  source 1..54: mol_type = other DNA
gttagttgatgtagtgttcaagacccaggaacttcagaagtctcgtgggctcgg

SEQ ID NO: 76 - DNA, 54 bp, synthetic construct
  misc_feature 1..54: note = ont-BC64
  source 1..54: mol_type = other DNA
gttagttgatgtagtgaaagtcgatgaacggtgtctgtcgtctcgtgggctcgg

SEQ ID NO: 77 - DNA, 54 bp, synthetic construct
  misc_feature 1..54: note = ont-BC65
  source 1..54: mol_type = other DNA
gtctcgtccgctcggccttgtctggaggaagactgagaagtctcgtgggctcgg

SEQ ID NO: 78 - DNA, 54 bp, synthetic construct
  misc_feature 1..54: note = ont-BC66
  source 1..54: mol_type = other DNA
gtctcgtccgctcgggaagttagaagccacaaggatcgggtctcgtgggctcgg

SEQ ID NO: 79 - DNA, 54 bp, synthetic construct
  misc_feature 1..54: note = ont-BC67
  source 1..54: mol_type = other DNA
tacattgatgcatggggtgagcacacgagtatgacaaacgtctcgtgggctcgg

SEQ ID NO: 80 - DNA, 54 bp, synthetic construct
  misc_feature 1..54: note = ont-BC68
  source 1..54: mol_type = other DNA
gtctcgtccgctcggccaccttcgtgtttgcttagattcgtctcgtgggctcgg

SEQ ID NO: 81 - DNA, 54 bp, synthetic construct
  misc_feature 1..54: note = ont-BC69
  source 1..54: mol_type = other DNA
gttagttgatgtagtagatcacatgaggctcggactgtagtctcgtgggctcgg

SEQ ID NO: 82 - DNA, 54 bp, synthetic construct
  misc_feature 1..54: note = ont-BC70
  source 1..54: mol_type = other DNA
gttagttgatgtagtacactccattcgtaggatctcggtgtctcgtgggctcgg

SEQ ID NO: 83 - DNA, 54 bp, synthetic construct
  misc_feature 1..54: note = ont-BC71
  source 1..54: mol_type = other DNA
gtctcgtccgctcggctgttactacctgatgctcccagggtctcgtgggctcgg

SEQ ID NO: 84 - DNA, 54 bp, synthetic construct
  misc_feature 1..54: note = ont-BC72
  source 1..54: mol_type = other DNA
gttagttgatgtagtgtcggtatggaagacagtcagctagtctcgtgggctcgg

SEQ ID NO: 85 - DNA, 54 bp, synthetic construct
  misc_feature 1..54: note = ont-BC73
  source 1..54: mol_type = other DNA
gtctcgtccgctcgggagggttctgtcatcctgtttcttgtctcgtgggctcgg

SEQ ID NO: 86 - DNA, 54 bp, synthetic construct
  misc_feature 1..54: note = ont-BC74
  source 1..54: mol_type = other DNA
gttagttgatgtagtagtggaagtgttgggatgcttgtagtctcgtgggctcgg

SEQ ID NO: 87 - DNA, 54 bp, synthetic construct
  misc_feature 1..54: note = ont-BC75
  source 1..54: mol_type = other DNA
gtctcgtccgctcggacaacagggttcatcacaatggtcgtctcgtgggctcgg

SEQ ID NO: 88 - DNA, 54 bp, synthetic construct
  misc_feature 1..54: note = ont-BC76
  source 1..54: mol_type = other DNA
gttagttgatgtagtgtccagggttgatgtaacaagcatgtctcgtgggctcgg

SEQ ID NO: 89 - DNA, 54 bp, synthetic construct
  misc_feature 1..54: note = ont-BC77
  source 1..54: mol_type = other DNA
gtctcgtccgctcgggttgtatccctgagaaacaggtcggtctcgtgggctcgg

SEQ ID NO: 90 - DNA, 54 bp, synthetic construct
  misc_feature 1..54: note = ont-BC78
  source 1..54: mol_type = other DNA
gttagttgatgtagtttctgattcaaaggttcggttgttgtctcgtgggctcgg

SEQ ID NO: 91 - DNA, 54 bp, synthetic construct
  misc_feature 1..54: note = ont-BC79
  source 1..54: mol_type = other DNA
gtctcgtccgctcggcagcagtgagaactatctccgagagtctcgtgggctcgg

SEQ ID NO: 92 - DNA, 54 bp, synthetic construct
  misc_feature 1..54: note = ont-BC80
  source 1..54: mol_type = other DNA
gttagttgatgtagtgaatcgctatcctatgttcatccggtctcgtgggctcgg

SEQ ID NO: 93 - DNA, 54 bp, synthetic construct
  misc_feature 1..54: note = ont-BC81
  source 1..54: mol_type = other DNA
gtctcgtccgctcggccgaaacaacttcacaagatgagggtctcgtgggctcgg

SEQ ID NO: 94 - DNA, 54 bp, synthetic construct
  misc_feature 1..54: note = ont-BC82
  source 1..54: mol_type = other DNA
gttagttgatgtagttagtcctggaactcgacataccgtgtctcgtgggctcgg

SEQ ID NO: 95 - DNA, 54 bp, synthetic construct
  misc_feature 1..54: note = ont-BC83
  source 1..54: mol_type = other DNA
gtctcgtccgctcggttcgaccttacctagatcaagccagtctcgtgggctcgg

SEQ ID NO: 96 - DNA, 54 bp, synthetic construct
  misc_feature 1..54: note = ont-BC84
  source 1..54: mol_type = other DNA
gttagttgatgtagttggcacaggttctaggtccactacgtctcgtgggctcgg

SEQ ID NO: 97 - DNA, 54 bp, synthetic construct
  misc_feature 1..54: note = ont-BC85
  source 1..54: mol_type = other DNA
gtctcgtccgctcgggatcatccaactaactcctccgttgtctcgtgggctcgg

SEQ ID NO: 98 - DNA, 54 bp, synthetic construct
  misc_feature 1..54: note = ont-BC86
  source 1..54: mol_type = other DNA
gtctcgtccgctcggtacttacgcttgttgggatcacctgtctcgtgggctcgg

SEQ ID NO: 99 - DNA, 54 bp, synthetic construct
  misc_feature 1..54: note = ont-BC87
  source 1..54: mol_type = other DNA
gtctcgtccgctcggcctccctaacaacaggagcatgtagtctcgtgggctcgg

SEQ ID NO: 100 - DNA, 54 bp, synthetic construct
  misc_feature 1..54: note = ont-BC88
  source 1..54: mol_type = other DNA
gttagttgatgtagtctgcttcggatcggtagtagaagagtctcgtgggctcgg

SEQ ID NO: 101 - DNA, 54 bp, synthetic construct
  misc_feature 1..54: note = ont-BC89
  source 1..54: mol_type = other DNA
gtctcgtccgctcggcaactagccaaacattgatgctgtgtctcgtgggctcgg

SEQ ID NO: 102 - DNA, 54 bp, synthetic construct
  misc_feature 1..54: note = ont-BC90
  source 1..54: mol_type = other DNA
gttagttgatgtagtgcctcaaaccgtaccctctacatcgtctcgtgggctcgg

SEQ ID NO: 103 - DNA, 54 bp, synthetic construct
  misc_feature 1..54: note = ont-BC91
  source 1..54: mol_type = other DNA
tacattgatgcatggagtagcgtgagttcctatggagccgtctcgtgggctcgg

SEQ ID NO: 104 - DNA, 54 bp, synthetic construct
  misc_feature 1..54: note = ont-BC92
  source 1..54: mol_type = other DNA
gtctcgtccgctcggggtcctgtatctttccactcacaagtctcgtgggctcgg

SEQ ID NO: 105 - DNA, 54 bp, synthetic construct
  misc_feature 1..54: note = ont-BC93
  source 1..54: mol_type = other DNA
gtctcgtccgctcggcccaagtctgaagtgatggaaactgtctcgtgggctcgg

SEQ ID NO: 106 - DNA, 54 bp, synthetic construct
  misc_feature 1..54: note = ont-BC94
  source 1..54: mol_type = other DNA
gttagttgatgtagtgtaggtggcagtttgaggacaatcgtctcgtgggctcgg

SEQ ID NO: 107 - DNA, 54 bp, synthetic construct
  misc_feature 1..54: note = ont-BC95
  source 1..54: mol_type = other DNA
gtctcgtccgctcggaagtccattcttcttccagacagggtctcgtgggctcgg

SEQ ID NO: 108 - DNA, 54 bp, synthetic construct
  misc_feature 1..54: note = ont-BC96
  source 1..54: mol_type = other DNA
tacattgatgcatggatggtggactctatgaccgttcaggtctcgtgggctcgg

SEQ ID NO: 109 - DNA, 24 bp, synthetic construct
  misc_feature 1..24: note = NB09
  source 1..24: mol_type = other DNA
aaccaagactcgctgtgcctagtt

SEQ ID NO: 110 - DNA, 24 bp, synthetic construct
  misc_feature 1..24: note = NB10
  source 1..24: mol_type = other DNA
gagaggacaaaggtttcaacgctt

SEQ ID NO: 111 - DNA, 24 bp, synthetic construct
  misc_feature 1..24: note = NB11
  source 1..24: mol_type = other DNA
tccattccctccgatagatgaaac

SEQ ID NO: 112 - DNA, 24 bp, synthetic construct
  misc_feature 1..24: note = NB12
  source 1..24: mol_type = other DNA
tccgattctgcttctttctacctg

SEQ ID NO: 113 - DNA, 41 bp, synthetic construct
  misc_feature 1..41: note = spacer-barcode-linker
  source 1..41: mol_type = other DNA
tacagacgactacaaacggaatcgagtctcgtgggctcgga

SEQ ID NO: 114 - DNA, 82 bp, synthetic construct
  misc_feature 1..82: note = Corrupted spacer or linker
  source 1..82: mol_type = other DNA
cgtccgctcggcacaaagacaccgacaactttcttgtctcgtgggctcgggtctcgtccgctcggcacaaagacaccgacaa

(19) United States
(12) Patent Application Publication
(10) Pub. No.: US 2026/0245657 A1
(43) Pub. Date: Aug. 20, 2026

(54) A COMPUTER IMPLEMENTED METHOD FOR IDENTIFYING, IF PRESENT, A PRESELECTED GENETIC DISORDER

(71) Applicant: gMendel ApS, Kobenhavn O (DK)

(72) Inventors: Christos KYRIAKIDIS, Kobenhavn O (DK); Christopher James Luscher, Kobenhavn O (DK); Gjorgji MADJAROV, Kobenhavn O (DK); Aleksandar NIKOV, Kobenhavn O (DK); Zoran VELKOSKI, Kobenhavn O (DK)

(21) Appl. No.: 18/870,205

(22) PCT Filed: Jun. 1, 2023
(86) PCT No.: PCT/EP2023/064684

§ 371 Date: Nov. 27, 2024

(30) Foreign Application Priority Data
Jun. 1, 2022 (GR) 20220100462

(51) Int. Cl. G16B 20/20; C12Q 1/6806; C12Q 1/686; C12Q 1/6869; G16B 30/10; G16B 50/30

(52) U.S. Cl. CPC G16B 20/20; C12Q 1/6806; C12Q 1/686; C12Q 1/6869; G16B 30/10; G16B 50/30

(57) ABSTRACT

A computer implemented method is provided for identifying, if present, a preselected chromosomal aberration, features, such as patterns, in the nucleotide sequence and/or mutated nucleotide sequence in a sample obtained from a subject, such as a human being, animals, such as dogs, horses, cows. The method is based on a plurality of DNA samples each originating from different subjects and/or a plurality of DNA samples representing replicas for a single subject. Preferred embodiments optimize the process from genetic material by combining next generation sequencing technology and state-of-the-art data-driven modelling and machine learning to provide a fast and cost-effective platform with a connected diagnostics compute module and status and control interface.

### FIELD OF THE INVENTION

The present invention relates to a computer implemented method for identifying, if present, a preselected chromosomal aberration, features, such as patterns, in the nucleotide sequence and/or mutated nucleotide sequence in a sample obtained from a subject, such as a human being, animals, such as dogs, horses, cows, the method is based on a plurality of DNA samples each originating from different subjects and/or a plurality of DNA samples representing replicas for a single subject. Preferred embodiments optimizes the process from genetic material by combining next generation sequencing technology and state-of-the-art data-driven modelling and machine learning to provide a fast and cost-effective platform with a connected diagnostics compute module and status and control interface.

### BACKGROUND OF THE INVENTION

Genetic disorders (GDs) affect nearly 350 million families with children, worldwide Timely and accurate diagnosis and treatment remain a great challenge for patients, doctors, and healthcare systems. The commonly used methods for genetic disorder detection are prohibitively costly. Moreover, the whole process of genetic disorder detection is usually followed by multiple misdiagnoses which increase the time of the correct diagnosis, with a lifetime cost surpassing Euro 2.5 million. Also, it requires scarce expert knowledge to unlock the genomic insights that can be obtained with the current mainstream technology.

Current methods that are used for genetic sequencing are designed towards a target aberration level (such as chromosome, gene, or base-pair level). Both microarray and qPCR methods may be used to detect gene expression and chromosomal abnormalities while Sanger sequencing is designed to detect genetic aberrations at the single base pair level.

The massive leap in DNA-sequencing methods made within the past decade heralded the inevitable decline of many old-fashioned DNA fingerprint-based typing methods. A single HiSeq X instrument (IIlumina) has a capacity to sequence about 35,000 average size bacterial genomes with 100 times coverage in a single run (Illumina.com). Yet, notwithstanding the immense potential, this technology is still not meant for fast, routine, and cost-effective diagnosis. It is mainly due to the high equipment cost, low flexibility requiring collection of multiple samples (from dozens to thousands depending on the platform), relatively long runtime, and complex data analysis.

Further, some DNA-sequencing methods are short read based and potentially some information may be lost in the bioinformatics analysis by that alignments are artificially biased to specific regions.

On the other hand, the portable, USB powered MinION offered by Oxford Nanopore Technologies (ONT) is so far the cheapest (~$1000) sequencing platform on the market. Its main advantage besides the price is the possibility to generate ultra-long reads with the longest ones crossing 1 Mb. Nonetheless, there are two main reasons why ONT has not yet become the first choice for sequencing. First is the relatively high basecalling error rate, such as 1% to 5% of a single DNA molecule and second, a relatively low throughput compared to many other platforms.

Hence, an improved method of sequencing and analysing DNA samples would be advantageous, and in particular a more efficient and/or reliable method and analysing of DNA samples would be advantageous.

### OBJECT OF THE INVENTION

It is a further object of the present invention to provide an alternative to the prior art for diagnosis of GDs.

In particular, it may be seen as an object of the present invention to provide a method and system that solves the above mentioned problems of the prior art.

### SUMMARY OF THE INVENTION

In a first aspect, the invention relates to a computer implemented method for identifying, if present, a preselected chromosomal aberration, features, such as patterns, in a target sequence and/or mutated target sequence in a DNA sample obtained from a subject, the method is based on a plurality of DNA samples each originating from different subjects and/or a plurality of DNA samples representing replicas for a single subject, the method preferably comprises
providing a DNA sample from multiple subjects and/or from DNA samples representing replicas from a single subject;
tagging each target sequence with a molecular barcode, wherein the tagging is a multilevel multiplexing tagging in which the plurality of target sequence are grouped into a number of groups each having a unique group barcode and each sample within a group is having a unique sample barcode within said group;
after said tagging, sequencing said tagged target sequences in parallel by use of a sequencing platform to provide nucleotide sequences;
de-multiplexing the nucleotide sequences on the basis of said barcodes and comparing by use of a computer nucleotide sequences, represented in a data record, with a reference nucleotide sequence, represented in a data record, and if the comparison shows a match between said target sequence of the nucleotide sequence and said reference nucleotide sequence, recording the group barcode and the sample barcode for the matched de-multiplexed nucleotide sequence.

In a second aspect, the invention relates to a computer implemented method for identifying, if present, a preselected chromosomal aberration, features, such as patterns, in a target sequence and/or mutated target sequence in a DNA sample obtained from a subject, the method is based on a plurality of DNA samples each originating from different subjects and/or a plurality of DNA samples representing replicas for a single subject, the method comprises
providing a DNA sample from multiple subjects and/or from DNA samples representing replicas from a single subject;
amplifying one more target sequences in said DNA samples, thereby providing amplified DNA samples;
tagging each target sequence with a molecular barcode, wherein the tagging is a multilevel multiplexing tagging in which:
   the amplified DNA samples are grouped into a number of groups each having a unique group barcode; and
   each sample and its one more target sequences within a group is having the same unique sample barcode within said group;
after said tagging, sequencing said tagged target sequences in parallel using single cell sequencing, such as a next generation sequencing platform, such as a nanopore DNA sequencing to provide nucleotide sequences;
de-multiplexing in real-time and/or latency bounded by which the de-multiplexing and the comparing are carried out when nucleotides are provided by the sequencing platform the nucleotide sequences on the basis of said barcodes and comparing by use of a computer nucleotide sequences, represented in a data record, with a reference nucleotide sequence, represented in a data record, and if the comparison shows a match between said target sequence of the nucleotide sequence and said reference nucleotide sequence, recording the group barcode and the sample barcode for the matched de-multiplexed nucleotide sequence.

The invention provides in preferred embodiments an automated, real-time, accurate, reliable, and cost-effective technology for simultaneous identification of genetic diseases and/or disorders on hundreds of different genetic sources.

Preferred embodiments of the invention have suggested substantial improvements towards the standardisation, automation cost-reduction and/or higher reliability of the identification process of genetic diseases and/or disorders using the next generation sequencing and machine learning technologies. Further on, preferred embodiments of the invention may enable large-scale parallelization of the analysis process without any assistance of highly specialised personnel.

Words used herein are used in a manner being ordinary to a skilled person. Some of these worded are elucidated here below:

### Primer

In the present context, the term “primer” is to be understood as a short, single-stranded DNA sequence used in the polymerase chain reaction (PCR) technique. In a PCR method, a pair of primers may be used to hybridize with the sample DNA and define the region of the DNA that will be amplified.

### Multiplexing/Demultiplexing

Multiplexing and Demultiplexing are preferably used to reference a process where multiple sources of genetic material are processed simultaneously (Multiplexing) and separated up into individual sources post-seceding (Demultiplexing).

### Tagging

In the present context, the term “tagging” refers to the process of attaching a molecular barcode to a target sequence in order to mark the target sequence with a unique code for later identification purposes.

Tagging may be performed by adding a barcode using PCR-technique as commonly known to the persons skilled in the art. Alternatively, barcodes may be attached by chemical modifications such as ligation.

### Molecular Barcode

In the present context, the term “molecular barcode” refers to composite barcode comprising a unique sample barcode and a unique group barcode. The molecular barcode may also comprise a linker and a spacer. Depending on the multiplexing level, it might comprise additional sequences to allow more samples to be analysed simultaneously.

Preferably, the molecular barcode is a short artificial section of DNA attached directly or indirectly to individual target samples.

In one embodiment, the sample barcode is around 6-60 nucleotides, such as 6-55 nucleotides, like 6-50 nucleotides, such as 10-40 nucleotides, like 20-30 nucleotides. In a further embodiment, the group barcode is around 6-60 nucleotides, such as 6-55 nucleotides, like 6-50 nucleotides, such as 10-40 nucleotides, like 20-30 nucleotides.

In a still further embodiment, the molecular barcode could also comprises a unique organization barcode. The organization barcode is preferably around 6-60 nucleotides, such as 6-55 nucleotides, like 6-50 nucleotides, such as 10-40 nucleotides, like 20-30 nucleotides.

### Linker

In the present context, the term “linker”, refers to a section of DNA, which may be comprised in the molecular barcode and arranged in connection with one or more barcodes of the molecular barcode.

The linker may be arranged at the 3′ and/or 5′ end of the target sequence and one or more barcodes of the molecular barcode.

In one embodiment, the linker is a palindromic sequence comprised in the 5′ end of both, forward and reverse primers used for tagging the target sequence with a barcode e.g. in PCR1.

The linker may be arranged in direct continuation of the barcode such as in direct continuation of the sample barcode.

In one embodiment, the linker is around 3-30 nucleotides, such as 5-25 nucleotides, like 10-20 nucleotides, such as 13-17 nucleotides.

### Spacer

In the present context, the term “spacer”, refers to a section of DNA, which may be comprised in the molecular barcode and arranged in connection with one or more barcodes. The spacer may be on the 3′ or 5′ end of the target sequence. The spacer may be arranged in direct continuation of the sample barcode.

Preferably, the sample, group and/or organization barcode is arranged between the spacer and linker as disclosed in SEQ ID NO: 113. However, the spacer and linker may be arranged both on either the 3′ or 5′ end of the molecular barcode.

In one embodiment, the spacer is around 3-30 nucleotides, such as 5-25 nucleotides, like 10-20 nucleotides, such as 13-17 nucleotides.

### Subject

The term “subject” comprises humans of all ages, mammals in general, including commercially relevant mammals, such as cattle, pigs, horses, sheep, goats, mink, ferrets, hamsters, cats and dogs, as well as birds. Preferred subjects are humans. In one embodiment, the subject is an embryo.

### DNA Samples

In the present context, “DNA sample(s)” refer to one or more samples of DNA obtained from a subject. The DNA sample comprises a target sequence. In one embodiment, the DNA sample is cfDNA. As an example, cfDNA from an embryo may be obtained via a sample from the mother enabling a pre-natal test to be performed.

The sample may be obtained from tissues or fluids such as blood, serum, plasma, amniotic fluid, saliva and/or urine.

### Target Sequence

In the present context, “target sequence” is to be understood as the part of the DNA sample comprising the gene, part of the gene, genetic sequence or part of genetic sequence known to be affected by mutation(s), chromosomal aberration(s), feature(s) or pattern(s) relevant for the specific genetic disorder of interest i.e. known to cause the genetic disorder.

The target sequence is tagged with a molecular barcode according to the present invention resulting in a tagged target sequence.

### Genetic Disorder

A genetic disorder is a health problem caused by one or more abnormalities in the genome. It can be caused by a mutation in a single gene or multiple genes or by a chromosomal abnormality.

Examples of genetic disorders are Turner syndrome, Prader-Wili syndrome, Angelman syndrome, Trisomy 18, Trisomy 13 and Klinefelter syndrome.

### Amplification

In the present context, “amplification” refers to the process of massive replication of genetic material, such as a gene or DNA sequence e.g. by means of polymerase chain reaction (PCR).

According to the present invention, the target sequence comprised in the DNA sample may be amplified.

### (Next-Generation) Sequencing

In the present context, sequencing refers to the process of determining the nucleic acid sequence—the order of nucleotides in DNA.

Next-generation sequencing (NGS) is typically highly scalable. An example of a NGS platform is the GridION x5 DNA sequencing device from Oxford Nanopore Technologies. Other examples of NGS platforms are Illumina-based ones or ThermoFisher sequencers as well as technology from PacBio.

### Nucleotide Sequences

In the present context, “nucleotide sequences” are to be understood as the determined nucleic acid sequence obtained by sequencing of the tagged target sequence and comprises the target sequence and the molecular barcode.

### Reference Nucleotide Sequence

In the present context, the term “reference nucleotide sequence” is to be understood as a nucleotide sequence known to express the mutation(s), chromosomal aberration(s), feature(s) or pattern(s) relevant for the specific genetic disorder of interest i.e. known to cause the genetic disease.

### Match Between Target Sequence of Nucleotide Sequence and Reference Nucleotide Sequence

The match between the target sequence of the nucleotide sequence and the reference nucleotide sequence should be at least 95% sequence identity, such as 96% sequence identity, like 97% sequence identity, such as 98% sequence identity, like 99% sequence identity, such as 99.5% sequence identity, like 99.8% sequence identity, such as 99.9% sequence identity, like 100% sequence identity. In one embodiment, the match is 100% sequence identity i.e. the sequences are identical.

### Sequence Identity

In the present context, the term “sequence identity”, refers to the sequence identity between genes or proteins at the nucleotide, base or amino acid level, respectively. Specifically, a DNA and a RNA sequence are considered identical if the transcript of the DNA sequence can be transcribed to the identical RNA sequence.

Thus, in the present context “sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned.

To determine the percent identity of two amino acid sequences or of two nucleic acids, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=#θ of identical positions/total # of positions (e.g., overlapping positions)×100). In one embodiment, the two sequences are the same length.

In another embodiment, the two sequences are of different length and gaps are seen as different positions. One may manually align the sequences and count the number of identical amino acids. Alternatively, alignment of two sequences for the determination of percent identity may be accomplished using a mathematical algorithm. Such an algorithm is incorporated into the NBLAST and XBLAST programs of (Altschul et al. 1990). BLAST nucleotide searches may be performed with the NBLAST program, score=100, wordlength=12, to obtain nucleotide sequences homologous to a nucleic acid molecules of the invention. BLAST protein searches may be performed with the XBLAST program, score=50, wordlength=3 to obtain amino acid sequences homologous to a protein molecule of the invention.

To obtain gapped alignments for comparison purposes, Gapped BLAST may be utilized. Alternatively, PSI-Blast may be used to perform an iterated search, which detects distant relationships between molecules. When utilising the NBLAST, XBLAST, and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g. “scoring matrix” and “gap penalty” may be used for alignment. In the context of the present invention, the BLASTN and PSI BLAST default settings may be advantageous.

The percent identity between two sequences may be determined using techniques similar to those described above, with or without allowing gaps. In calculating percent identity, only exact matches are counted. An embodiment of the present invention thus relates to sequences of the present invention that has some degree of sequence variation.

### BRIEF DESCRIPTION OF THE FIGURES

The present invention and in particular preferred embodiments thereof will now be described in more details with regard to the accompanying figures. The figures show ways of implementing the present invention and are not to be construed as being limiting to other possible embodiments falling with the scope of the attached claim set.

FIG. 1 schematically illustrating process steps carried out in processing DNA samples according to a preferred embodiment;

FIG. 2 schematically illustrating on a DNA level process steps in processing DNA samples according to a preferred embodiment;

FIG. 3 is a flowchart illustrating various steps involved in multilevel multiplexing and de-multiplexing according to a preferred embodiment, kindly observe that the number of samples (96) and four parallel processes are non-limiting examples. In some embodiments, the process run in parallel several flowcells, and the number of samples (e.g. 96) for a first level multiplexing are multiplexed e.g. four times within one flowcell;

FIG. 4 schematically illustrates steps involved in a multilevel multiplexing according to a preferred embodiment; also for this embodiment, the number of sample (96) and two parallel processes are non-limiting examples; however, FIG. 4 may be viewed as providing further details as to the embodiment shown in FIG. 3;

FIG. 5 schematically illustrates an embodiment of two-level barcoding used in preferred embodiments of a multilevel multiplexing; kindly observe that although the barcodes are illustrated as positioned at the 3′-end of a nucleotide sequence, the barcodes may be positioned in other positions;

FIG. 6 schematically illustrates an embodiment of a three-level barcoding used in preferred embodiments of a multilevel multiplexing; again, kindly observe that although the barcodes are illustrated as positioned at the 5′-end of a nucleotide sequence, the barcodes may be positioned in other positions.

FIG. 7 schematically illustrates process involved in setting up (training) and use of a machine learning binary classifier in a preferred embodiment of the invention;

### DETAILED DESCRIPTION OF PREFERRED EMBODIMENTS

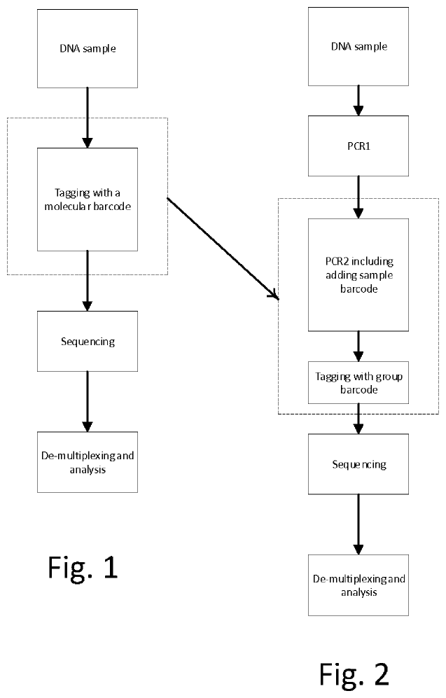

Reference is made to FIG. 1 schematically illustrating process steps carried out in processing DNA samples according to a preferred embodiment. As is apparent from the embodiment of FIG. 1, processing starts with DNA extraction and ends with clinical reporting. In FIG. 1, the clinical reporting is considered to be within the analysis process. Such an analysis process may involve using an artificial intelligence or machine learning process to interpret results. However, the invention is not limited to such start and end processes. The processing of DNA comprises in the illustrated embodiments, DNA extraction, PCR amplification, sequencing, analysis of the sequencing results, interpretation and reporting based on the analysis of the sequencing results, and clinical management. The clinical management may comprise providing the result and the interpretation thereof to a user, such as an end user.

FIG. 2 illustrates a method according to a preferred embodiment. The method includes obtaining DNA-samples and carrying out a first PCR (PCR1) with the purpose of amplifying a target sequence of DNA. Subsequently, a second PCR (PCR2) is carried out including tagging with a group barcode which will be further detailed below. Following the tagging is a sequencing, a demultiplexing and an analysis which may be as disclosed above.

In preferred embodiments, the analysis and classification process of the DNA sequence is obtained by the Oxford Nanopore device. The process comprises multiple data pre-processing steps, classification, data post-processing and final decision generation. The pre-processing steps are preferably performed to improve the data quality and obtain better data representation.

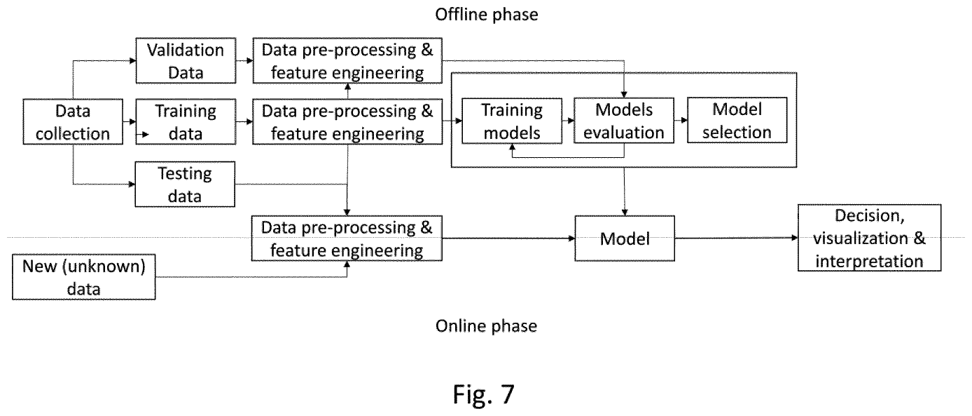

The second phase (classification) may be represented by a complex, specifically designed multi-level decision architecture that in preferred embodiments utilizes Machine Learning algorithms and recent research in sequence analysis. It may employ multiple base and conceptual models to improve the predictive performance, efficiency and their understandability. Different, tailored made architectures may be employed for some or even every step in the learning process (feature extraction, feature selection, dimensionality reduction, etc.) and, learned and validated using carefully selected genome sequences that satisfy rigorous quality criteria. Their individual decisions may be combined using a hybrid model that enhances the predictive performance. The multi model fusion strategy, preferably, built on top of the individual models may combine their outcome into a singular final decision. To this, FIG. 7 details process and steps involved in a preferred embodiment based on a binary classifier.

As disclosed herein, preferred embodiments of the invention may be considered to a computer implemented method for identifying, if present, a preselected chromosomal aberration, features, such as patterns, in the nucleotide sequence and/or mutated nucleotide sequence in a sample obtained from a subject, such as a human being, animals, such as dogs, horses, cows. It is noted that the computer implemented features are related to data processing whereas the processing of DNA samples is carried out by suitable devices configured to carry out the processing desired.

Preferred embodiments of the method is based on a plurality of DNA samples each originating from different subjects and/or a plurality of DNA samples representing replicas for a single subject. The samples are obtained in a manner being well known to the skilled person and comprises providing a DNA sample from multiple subjects and/or from DNA samples representing replicas from a single subject.

In many embodiments, the DNA samples are samples from multiple subjects and the following disclosure will be focused towards such multiple samples. However, when replicas are used, the procedures disclosed are similar such as identical.

After the DNA samples are provided, tagging of the samples is carried out. The tagging comprising tagging each DNA sample with a molecular barcode. The tagging is a multilevel multiplexing tagging in which the plurality of DNA samples are grouped into a number of groups each having a unique group barcode and each sample within a group is having a unique sample barcode within said group.

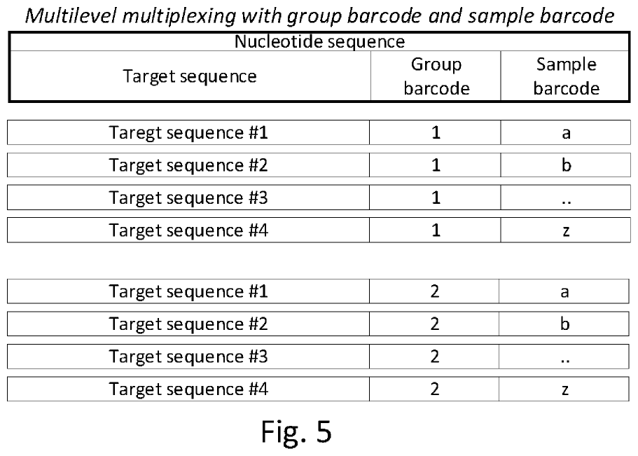

An embodiment of a multilevel multiplexing is illustrated in FIG. 5. As indicated in FIG. 5, four nucleotide sequences (denoted in FIG. 5 by nucleotide sequence #1-#4) are multilevel multiplexed. As apparent from FIG. 5, the four nucleotide sequences are divided into two groups where each group has a unique group barcode. After the individual nucleotide sequences have been tagged with a sample barcode, each sequence is tagged with a group barcode identifying the group which the sequence forms a member of.

Accordingly, in preferred embodiments, target sequence tagged with a molecular barcode comprising a group barcode and a sample barcode may symbolically be summarised as:

(target sequence)+(group barcode)+(sample barcode)

Kindly observe that the order in which target sequence, group barcode and sample barcode appear is only for illustration purpose and the order in which the different elements appears may be different in other embodiments.

Accordingly, in preferred embodiments, target sequence tagged with a molecular barcode comprising a group barcode and a sample barcode may symbolically be summarised as:

(target sequence)+(organizational barcode)+(group barcode)+(sample barcode)

Kindly observe that the order in which target sequence, organizational barcode, group barcode and sample barcode appear is only for illustration purpose and the order in which the different elements appears may be different in other embodiments.

Accordingly, in preferred embodiments, target sequence tagged with a molecular barcode comprising a group barcode and a sample barcode may symbolically be summarised as:

(target sequence)+(spacer)+(group barcode)+(linker)+(sample barcode)

Kindly observe that the order in which target sequence, spacer, group barcode, linker and sample barcode appear is only for illustration purpose and the order in which the different elements appears may be different in other embodiments.

Further, the terminology used herein distinguish between sample, which although comprising nucleotide sequences, and nucleotide sequence. Herein “sample” or DNA sample is used to reference to the sample prior to tagging “nucleotide sequence” is used to reference the outcome of the sequencing.

After the tagging, the DNA samples are sequenced, typically in parallel, by use of a sequencing platform to provide nucleotide sequences. The nucleotide sequences originating from the sequencing are the sequences to be analysed. It is noted that sequencing includes sequencing of the barcode.

The analysis of the nucleotide sequences obtained by the sequencing process comprises a de-multiplexing the nucleotide sequences on the basis of the barcodes and comparing by use of a computer the target sequence part of the nucleotide sequence with a reference nucleotide sequence. In this process, the computer evaluated whether a nucleotide sequence is similar to or identical to a reference nucleotide sequence.

Such a reference nucleotide sequence is preferably a nucleotide expressing a genetic disorder. If the comparison shows a match between the nucleotide sequence and said reference nucleotide sequence, the computer records the group barcode and the sample barcode for the matched de-multiplexed nucleotide sequence. As the sample barcode carries a unique code for the DNA sample, the DNA sample can be identified.

Preferred embodiments of the invention also comprising holding information such as storing in database information that links the sample barcode to the individual from which the DNA sample is provided. Thus, when the DNA sample has been identified, the individual from which the DNA sample comes from can be identified on the basis of the sample barcode.

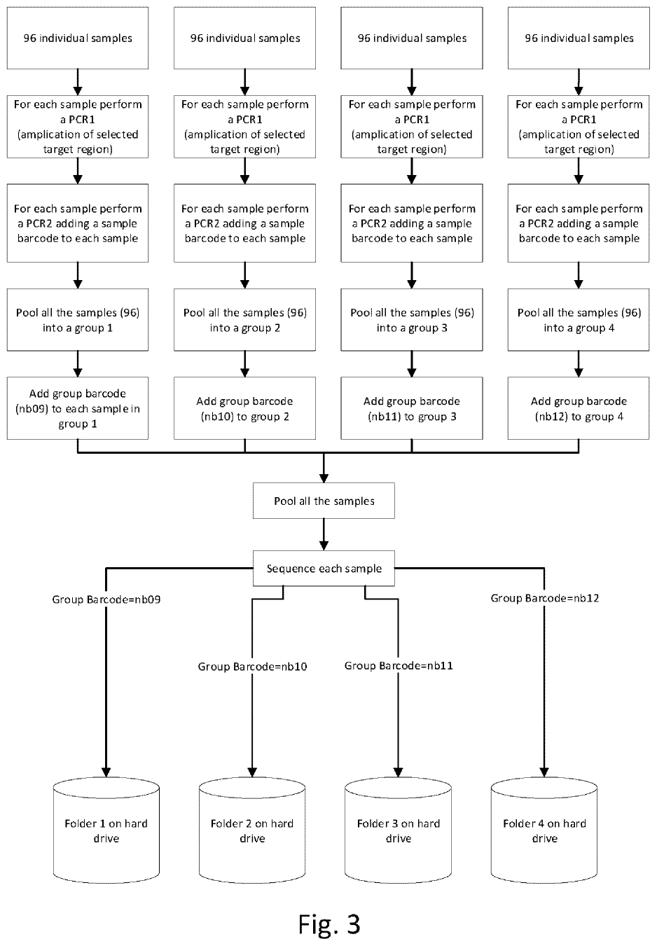

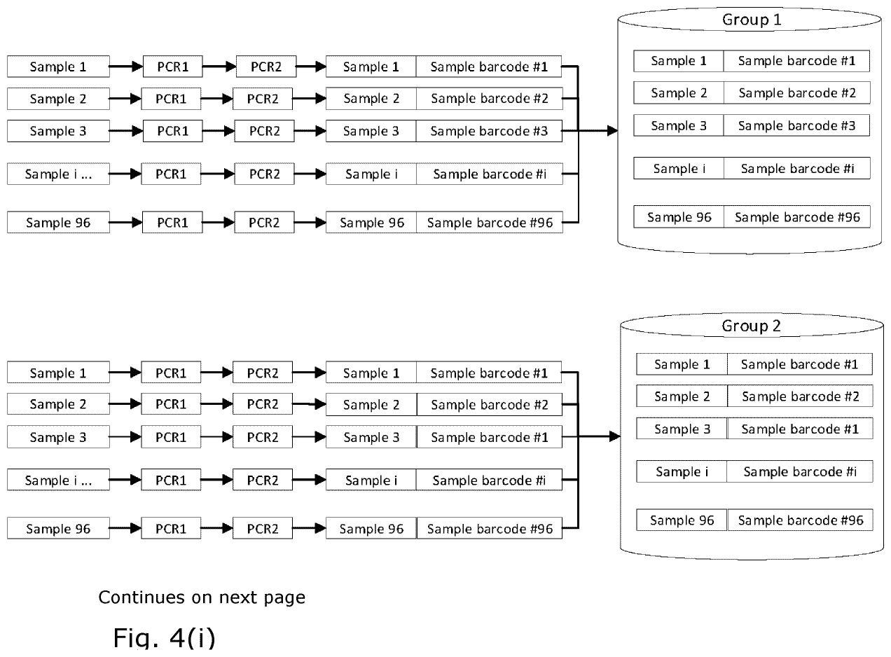

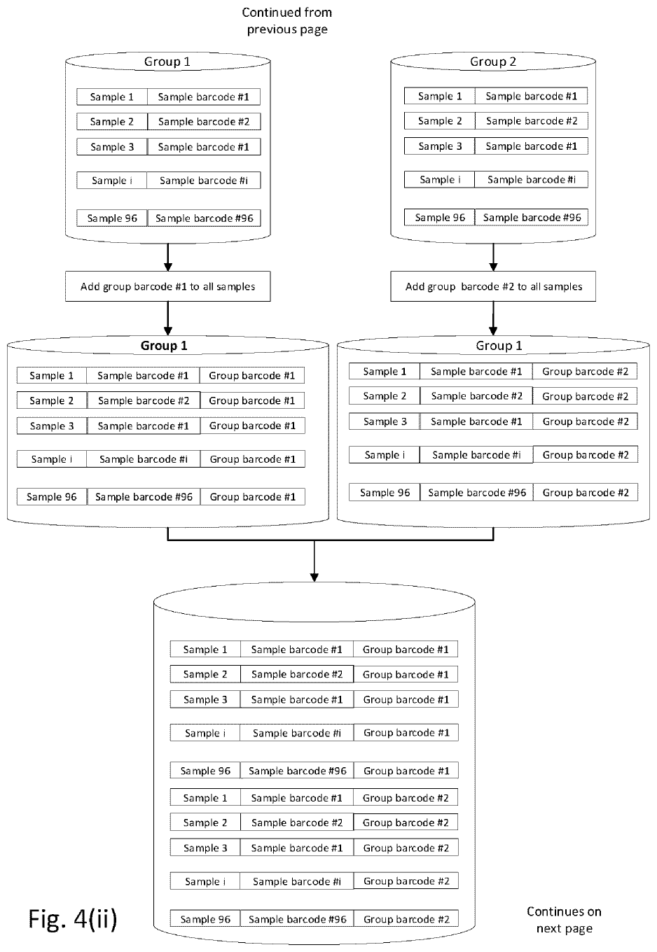

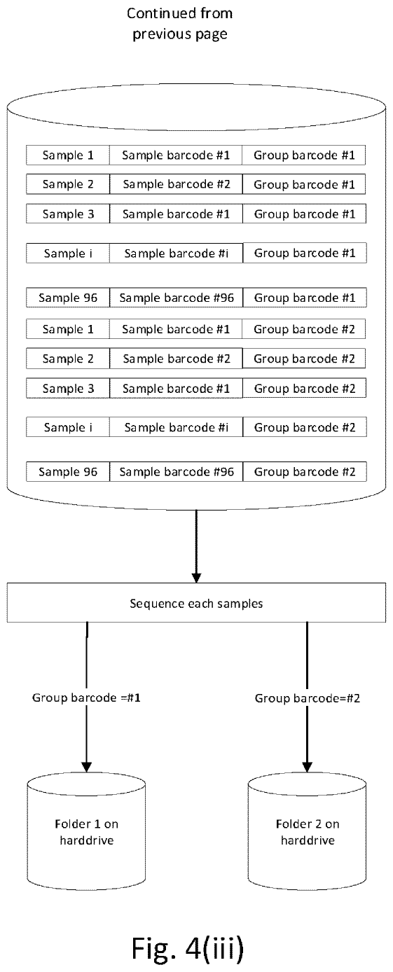

FIG. 3 provides some further details as a to preferred embodiment of the invention in which number of sample and number of parallel processing are non-limiting examples. As illustrated in FIG. 3, ninety-six individual DNA sample are provided. These ninety-six samples are grouped in four groups as illustrated by the four parallel flows in the upper part of the flow chart of FIG. 3—by this four groups each having 96 samples are provided. It is noted that the samples in each group are the same. For each DNA sample, a PCR1 is carried out. “PCR1” is used to distinguish this PCR process from a subsequent PCR2 process which is carried out after the first PCR1. PCR1 is used for target amplification and may be optimised and performed as commonly known to persons skilled in the art. As shown in FIG. 3, the subsequent PCR2 adds a first barcode to each sample. This first barcode is the sample barcode which is unique for each DNA sample (this may become more apparent from the description of FIG. 4). During the PCR1 and PCR2 the samples are kept separate from each other, and when the PCR2 has been applied to a sample, it is placed in the pool whereby the pool, at the end, comprising all ninety-six samples in one pool. Thus, in the illustrative example, four pools are provided which are referred to as groups.

After pooling into groups, a group barcode is added to each of the samples in each pool, where the group barcode is unique to the pool (group) in which the sample is present.

After the group barcode has been added to each sample, all the samples are pooled together. Thus, this pools comprising what may be referred to replicas of the original ninety-six sample, where each replica of a sample has the same sample barcode but different group barcode.

The pooled samples are the sequenced. Once an output is ready from the sequencer, the group barcode is identified and the output is stored in folder representing the group barcode identified. This process may be visualized as a sorting of the output from the sequencer where the output is sorted according to a respective group barcode. As an example, if the output contains the group barcode “nb10” it is stored in folder 2 on a hard drive. It is noted that storing in folder should be interpreted broadly as the storing may be implemented in many ways e.g. by tagging an output with a tag or meta data reflecting or representing group barcode, that is the outputs need not to be physically ordered in a folder. In general, this sorting may be seen as a re-grouping of the output into the same groups into which the DNA samples were groups prior to pooling and sequencing.

In an embodiment, this re-grouping forms part of the de-multiplexing, which further comprises identification of the sample barcode. However, in general de-multiplexing is preferably considered to comprise the steps of identifying the group barcode and the sample barcode in the output from the sequencer.

FIG. 4 provides a further description of the multilevel multiplexing. FIG. 4 details a similar procedure as disclosed in regards to FIG. 3; however in the example of FIG. 4 only two parallel PCR processes are carried out. As seen in FIG. 4, ninety-six samples are obtained and a PCR2 provides an individual sample barcode to each sample. As seen in FIG. 4, two groups are provided namely Group 1 and Group 2. The process may also be disclosed as PCR1 amplify target region and PCR2 amplify barcoded sample, where primers in PCR1 are specific to amplify target region and primers for PCR2 are specific to a particular barcode.

For each sample in each group a group barcode is added in correspondence with the specific group in which the sample is present. After that, all samples are pooled and subsequently sequenced and the output re-grouped.

It is to be emphasised that although FIGS. 3 and 4 illustrate parallel processing involved in PCR and addition of barcodes, the invention is not limited to parallel processing as sequential processing may be applied, although this may result in slowdown of the processing.

In some preferred embodiments, the de-multiplexing and the comparing of each of the nucleotide sequences are performed in real-time and/or latency bounded. Real-time and/or latency bounded preferably means that the de-multiplexing and the comparison are carried out when nucleotides are provided by the sequencing platform, that is preferably without any delay between provision of the nucleotides and the de-multiplexing and comparison. However, the invention is not limited to such real-time and/or latency bounded processes as it may be chosen to process e.g. the results in a batch processing.

The sequencing platform may be configured to provide sequencing results concurrently with the sequencing of one of said DNA sequence. In such embodiments, the method may be configured to abort further sequencing of a DNA sequence if the comparison shows said match. This may be implemented by using an abort-criteria according to which the sequencing is aborted if a predefined plurality of sequences, such as 10000 sequences, are matched per barcode or a disease is detected based on real-time analysis.

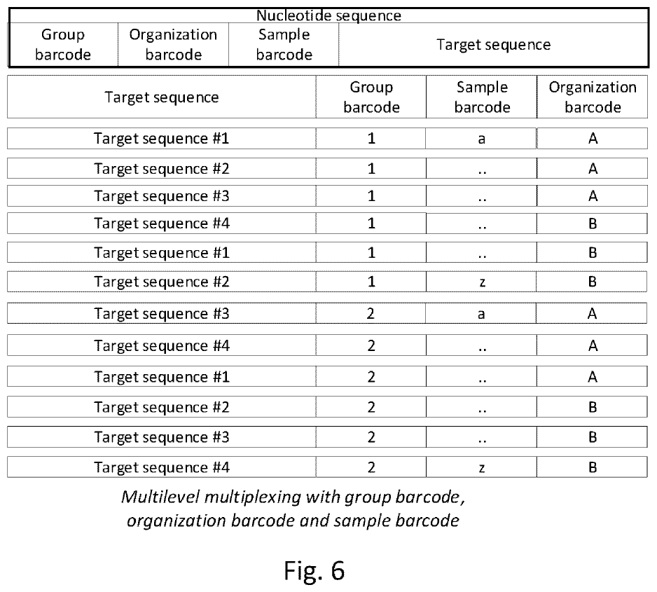

Reference is made to FIG. 6 showing a further embodiment of multilevel multiplexing. In the illustrated embodiment, the multilevel multiplexing further comprises organizing the DNA samples within one of the groups into at least two organizations. It is noted that organization is used in a broad meaning and refers to the samples are organized, thereby not necessarily referring to an organization from which the sample comes from. In such embodiments, organization has a unique organization barcode within the group, and the tagging further includes tagging said DNA samples with the organization barcodes. Such organization barcodes may e.g. be used in sorting, indexing and organization sample, which may be used e.g. for error detection where the sorting can be used to track back to identify an origin of an error.

In order to link a DNA sample to an individual, preferred embodiments of the invention further comprises storing in a barcode database, the barcodes used in the tagging where each barcode is tagged with information linking a specific barcodes to a specific individual. This may be done in numerous manner such as storing a record in a data structure comprising the identity of the individual and the barcode used for the individual. The identity of the individual may be a number, name or other identifiers allowing the identification of the individual. Thus, the de-multiplexing may further comprise performing a database look-up in the barcode database to retrieve said information linking a barcode to a specific individual. Once the individual has been identified, the information may be passed on to e.g. a medical doctor or other relevant personnel which then can act upon that an individual has been found to have a genetic disorder.

Today, sequencing may be carried out in parallel, which allows for a high throughput, and this is used in preferred embodiments of the invention by using a sequencing platform which is configured to parallel sequencing using single cell sequencing. Such platform are known as “a next generation sequencing platform”, and one particular preferred platform is a so-called nanopore DNA sequencing which will be further detailed in the section below detailing experiments.

In some preferred embodiments, the tagging further comprises addition of a molecular spacer and a molecular linker at a start or at an end of a DNA sequence.

The molecular spacer and molecular linker each comprises a known sequence of nucleotides and is advantageously used to located the barcode. Such location of the barcode is preferably considered to form part of the de-multiplexing which may comprise for a DNA sequence to be de-multiplexed the steps of:
searching from the start and/or the end of said DNA sequence to identify the molecular spacer and the molecular linker preferably by use of a sliding window. The sliding window refers to a give length of a DNA sequence to by compared, such as a length of 15 nucleotides. The sliding window is shifted along the DNA sequence e.g. with a stride of 1. The searching preferably comprises comparing a sequence of nucleotides of the DNA sequence to be de-multiplexed located within the sliding window with the known sequences of nucleotides (linker or spacer) and if a match is found for both the molecular linker and the molecular spacer then a nucleotide sequence in between the molecular spacer and molecular linker is assigned to be a barcode.

It is to be noted that the barcode may not necessarily be located between the spacer and linker, and in such scenarios the barcode may still be located by searching the DNA sequence.

The comparison may preferably include the process of evaluating a similarity measure providing e.g. a number indicating a level of a match. In some preferred embodiments, a Levenshtein Distance process is used within the sliding window. In such embodiments, a match may be considered to be present if the similarity measure is less than a predefined threshold, such as Levenshtein Distance is less than 10, such as less than 7, such as less than 5.

As detailed herein, a comparison between a reference nucleotide sequence and a nucleotide obtained by the sequencing. Numerous ways may be used to perform such a comparison and in preferred embodiments of the invention, comparing nucleotide sequences with a reference nucleotide sequence may be carried out by use of a basic local alignment search tool, such as BLAST, such as VSEARCH, such as USEARH, such as UCLUST.

Non-limiting examples on length of barcodes are:
group barcodes: a sequence of nucleotides with a length of around 6-60 nucleotides, such as 6-55 nucleotides, like 6-50 nucleotides, such as 10-40 nucleotides, like 20-30 nucleotides. In a further embodiment, the group barcode is around 6-60 nucleotides, such as 6-55 nucleotides, like 6-50 nucleotides, such as 10-40 nucleotides, like 20-30 nucleotides.
sample barcodes: a sequence of nucleotides with a length of around 6-60 nucleotides, such as 6-55 nucleotides, like 6-50 nucleotides, such as 10-40 nucleotides, like 20-30 nucleotides. In a further embodiment, the group barcode is around 6-60 nucleotides, such as 6-55 nucleotides, like 6-50 nucleotides, such as 10-40 nucleotides, like 20-30 nucleotides.
organization barcode: a sequence of nucleotides with a length of around 6-60 nucleotides, such as 6-55 nucleotides, like 6-50 nucleotides, such as 10-40 nucleotides, like 20-30 nucleotides. In a further embodiment, the group barcode is around 6-60 nucleotides, such as 6-55 nucleotides, like 6-50 nucleotides, such as 10-40 nucleotides, like 20-30 nucleotides.

### EXPERIMENTS CARRIED OUT BY USE OF PREFERRED EMBODIMENTS OF THE INVENTION

### Materials and Methods

### Samples

Extracted DNA samples were collected from Coriell Institute, at a quantity of 25 μg. Sample set involved Table 1. As for the male Turner Syndrome (Sample ID: NA02668) has remarks which notes that the individual involved has pseudohermaphrodie with several Turner Syndrome stigmata, mixed gonadal dysgenesis; 33% of cells with X, 45. All samples are pre-diagnostic from Coriell Institute, Table 1.

TABLE 1
samples

| Sample ID | Disorder |
|---|---|
| NA02668 | Turner Syndrome |
| NA10179 | |
| (from Coriell Institute) | |
| NA11386 | Prader-Willi Syndrome |
| NA11390 | |
| (from Coriell Institute) | |
| NA11517 | Angelman Syndrome |
| NA20375 | |
| (from Coriell Institute) | |
| NA02732 | Trisomy 18 |
| NG13074 | |
| (from Coriell Institute) | |
| NA03330 | Trisomy 13 |
| NG10292 | |
| (from Coriell Institute) | |
| NA03091 | Klinefelter Syndrome |
| NA03102 | |
| (from Coriell Institute) | |
| C-M | control male |
| C-F | control female |

### Primer Designing for Target Specific Regions

Cytogenetic chromosome analysis was build based upon primer design, which target a selected region of the chromosomes of interest. Primers where optimized through different temperature gradients, both individually and in a mixture of other selected primers targeting a different chromosome of interest. Table 2 represent the primers used in this experiment.

TABLE 2
primer + linker design for chromosomes of interest (from 5′ to 3′)

| | |
|---|---|
| | SEQ ID |
| Sequence* | NO |

Chromosome Y

| | | |
|---|---|---|
| Forward | 5′-GTCTCGTGGGCTCGGGATCTCTTCAGCGTGGGAGG-3′ | 1 |
| Reverse | 5′-GTCTCGTGGGCTCGGCTGAGAGGGTGAGGACAGGA-3′ | 2 |

Chromosome X

| | | |
|---|---|---|
| Forward | 5′-GTCTCGTGGGCTCGGGAGAGGGAGGAACGCATAGC-3′ | 3 |
| Reverse | 5′-GTCTCGTGGGCTCGGAGGGAGCAGAATTGAGGCAC-3′ | 4 |

Chromosome 21

| | | |
|---|---|---|
| Forward | 5′-GTCTCGTGGGCTCGGGTCCCCAGGTAACATCCACG-3′ | 5 |
| Reverse | 5′-GTCTCGTGGGCTCGGAGCTATAAGCCAAGGGACGC-3′ | 6 |

Chromosome 18

| | | |
|---|---|---|
| Forward | 5′-GTCTCGTGGGCTCGGGTCTAGAGTGGGAGGGGCTA-3′ | 7 |
| Reverse | 5′-GTCTCGTGGGCTCGGCCAGCTGGCTGAAATGAAGAA-3′ | 8 |

Chromsome 13

| | | |
|---|---|---|
| Forward | 5′-GTCTCGTGGGCTCGGTAGTTGAGGGGTGGCTTTGC-3′ | 9 |
| Reverse | 5′-GTCTCGTGGGCTCGGCCTACCTCCCTGTTCTCTGC-3′ | 10 |

Chromosome 15

| | | |
|---|---|---|
| Forward | 5′-GTCTCGTGGGCTCGGGTGGGCAGCTTCCAGTAGTT-3′ | 11 |
| Reverse | 5′-GTCTCGTGGGCTCGGCTGAGACTCGGGGTTTGCAT-3′ | 12 |

*The sequence comprises both primer sequence and linker sequence. The linker sequence is the 5′-end marked in italics.

Thus, primers as described in Table 2 (including a palindromic, linker region in 5′) were used for PCR1. The primers in Table 2 were used both as forward and as reverse primers for PCR2 hybridizing with the linker region at both ends.

### Amplification of Targeted Chromosomes, PCR1

The PCR amplification and interpretation of the results, were performed prior to obtaining the cytogenetic results. PCR1 consisted a total volume of 25 μL. Each 25 μL PCR reaction comprised 5 ng DNA, 12 μL PCRBIO UltraMix 400 rx, 2 μL primermix14 and 6 μL miliq. The primermix contained a mix of the primers as described in Table 2. Polymerase activation for PCR1 reaction was carried out at 95° C. for 5 minutes, where after 15 cycles of 95° C. for 30 seconds for initial DNA denaturation, 53° C. for 30 seconds for primer annealing along with primer extension at 72° for 45 seconds, and for final extension at 72° C. for 4 min. The amplified PCR1-product was cleaned with binding beads 8 μL pr. 25 μL.

### Clean-Up, PCR1 and PCR2

PCR product and was performed as followed: 8 μL of binding beads was added to each sample, incubated at room temperature (RT) at invitrogen, ThermoFisher, HulaMixer™ Sample Mixer. The mixed samples were then moved to magnetic rack until the mixed sampled appeared clear and a bead-pellet was formed. Without disturbing the bead-pellet, the supernatant was discarded, and bead-pellet was washed in 75% freshly prepared Ethanol (EtOH). This step was repeated. To ensure a total removal of EtOH, the sample is spun down, placed back on the magnetic rack where any leftover liquid was removed with a 15 μL pipette. The sample-tube is left on magnetic rack and open lid to air-dry for approximately 30 seconds to evaporate any lasted EtOH. The sample-tube is removed from the magnetic rack and the bead-pellet is resuspended in 20 μL of nuclease free water mixed by gently pipetting and incubated for 2 minutes at RT. The sample-tube is hereafter placed on magnetic rack, and when the liquid seems clear and a bead-pellet was formed, the liquid was transferred to a new Eppendorf tube.

### Amplification of Barcoded Regions of Chromosomes, PCR2

For PCR2, 10 μL of clean PCR1-product is transferred to a PCR-tube, along with 12 μL of PCRBIO UltraMix 400 rx and 2 μL of barcode (Table 3) and primers in Table 3 were used as both, forward and reverse primers in the PCR2-reaction. The PCR2-progamme was set as following: polymerase activation at 95° C. for 2 minutes, then 30 cycles of 95° C. for 20 seconds for denaturing DNA, 55° C. for 20 seconds for primer annealing, and 72° C. for 40 seconds for primer extension. Final extension was set to 72° C. for 4 minutes. After amplification, to ensure PCR2-products, the PCR2-samples separated on a gel. All PCR reactions were performed in thermal cycler PCRmax™ Alpha Cycler 1 Thermal Cycler.

TABLE 3
Primer sequences for PCR2 comprising linker, barcode and spacer

| Barcode ID | Sequence | SEQ ID NO. |
|---|---|---|
| ont-BC01 | 5′-GTCTCGTCCGCTCGGCACAAAGACACCGACAACTTTCTT | 13 |
| | GTCTCGTGGGCTCGG-3′ | |
| ont-BC02 | 5′-GTTAGTTGATGTAGTACAGACGACTACAAACGGAATCGA | 14 |
| | GTCTCGTGGGCTCGG-3′ | |
| ont-BC03 | 5′-GTCTCGTCCGCTCGGCCTGGTAACTGGGACACAAGACTC | 15 |
| | GTCTCGTGGGCTCGG-3′ | |
| ont-BC04 | 5′-GTTAGTTGATGTAGTTAGGGAAACACGATAGAATCCGAA | 16 |
| | GTCTCGTGGGCTCGG-3′ | |
| ont-BC05 | 5′-GTCTCGTCCGCTCGGAAGGTTACACAAACCCTGGACAAG | 17 |
| | GTCTCGTGGGCTCGG-3′ | |
| ont-BC06 | 5′-GTTAGTTGATGTAGTGACTACTTTCTGCCTTTGCGAGAA | 18 |
| | GTCTCGTGGGCTCGG-3′ | |
| ont-BC07 | 5′-GATATGATATAGATAAAGGATTCATTCCCACGGTAACAC | 19 |
| | GTCTCGTGGGCTCGG-3′ | |
| ont-BC08 | 5′-GTTAGTTGATGTAGTACGTAACTTGGTTTGTTCCCTGAA | 20 |
| | GTCTCGTGGGCTCGG-3′ | |
| ont-BC09 | 5′-GTCTCGTCCGCTCGGAACCAAGACTCGCTGTGCCTAGTT | 21 |
| | GTCTCGTGGGCTCGG-3′ | |
| ont-BC10 | 5′-GTTAGTTGATGTAGTGAGAGGACAAAGGTTTCAACGCTT | 22 |
| | GTCTCGTGGGCTCGG-3′ | |
| ont-BC11 | 5′-GTTAGTTGATGTAGTTCCATTCCCTCCGATAGATGAAAC | 23 |
| | GTCTCGTGGGCTCGG-3′ | |
| ont-BC12 | 5′-GTCTCGTCCGCTCGGTCCGATTCTGCTTCTTTCTACCTG | 24 |
| | GTCTCGTGGGCTCGG-3′ | |
| ont-BC13 | 5′-GTCTCGTCCGCTCGGTCACACGAGTATGGAAGTCGTTCT | 25 |
| | GTCTCGTGGGCTCGG-3′ | |
| ont-BC14 | 5′-TACATTGATGCATGGTCTATGGGTCCCAAGAGACTCGTT | 26 |
| | GTCTCGTGGGCTCGG-3′ | |
| ont-BC15 | 5′-GTTAGTTGATGTAGTCAGTGGTGTTAGCGAGGTAGACCT | 27 |
| | GTCTCGTGGGCTCGG-3′ | |
| ont-BC16 | 5′-TACATTGATGCATGGAGTACGAACCACTGTCAGTTGACG | 28 |
| | GTCTCGTGGGCTCGG-3′ | |
| ont-BC17 | 5′-GTCTCGTCCGCTCGGATCAGAGGTACTTTCCTGGAGGGT | 29 |
| | GTCTCGTGGGCTCGG-3′ | |
| ont-BC18 | 5′-GTTAGTTGATGTAGTGCCTATCTAGGTTGTTGGGTTTGG | 30 |
| | GTCTCGTGGGCTCGG-3′ | |
| ont-BC19 | 5′-GTTAGTTGATGTAGTATCTCTTGACACTGCACGAGGAAC | 31 |
| | GTCTCGTGGGCTCGG-3′ | |
| ont-BC20 | 5′-GTTAGTTGATGTAGTATGAGTTCTCGTAACAGGACGCAA | 32 |
| | GTCTCGTGGGCTCGG-3′ | |
| ont-BC21 | 5′-GTTAGTTGATGTAGTTAGAGAACGGACAATGAGAGGCTC | 33 |
| | GTCTCGTGGGCTCGG-3′ | |
| ont-BC22 | 5′-GTTAGTTGATGTAGTCGTACTTTGATACATGGCAGTGGT | 34 |
| | GTCTCGTGGGCTCGG-3′ | |
| ont-BC23 | 5′-GTCTCGTCCGCTCGGCGAGGAGGTTCACTGGGTAGTAAG | 35 |
| | GTCTCGTGGGCTCGG-3′ | |
| ont-BC24 | 5′-GTTAGTTGATGTAGTCTAACCCATCATGCAGAACTATGC | 36 |
| | GTCTCGTGGGCTCGG-3′ | |
| ont-BC25 | 5′-GTCTCGTCCGCTCGGCATTGCGTTGCATACCCAACTTAC | 37 |
| | GTCTCGTGGGCTCGG-3′ | |
| ont-BC26 | 5′-TACATTGATGCATGGATGAGAATGCGTAGTCGCTGTATG | 38 |
| | GTCTCGTGGGCTCGG-3′ | |
| ont-BC27 | 5′-GTCTCGTCCGCTCGGTGTAAGAGGTGAATCTAACCGTCG | 39 |
| | GTCTCGTGGGCTCGG-3′ | |
| ont-BC28 | 5′-GTTAGTTGATGTAGTGATACGGTGCCTTCTTAGGTTTCA | 40 |
| | GTCTCGTGGGCTCGG-3′ | |
| ont-BC29 | 5′-GTTAGTTGATGTAGTGGTCTGTCAACCCAAGGTGTCTAG | 41 |
| | GTCTCGTGGGCTCGG-3′ | |
| ont-BC30 | 5′-GTTAGTTGATGTAGTTGGGTCGAAGTAGATCCTCACTGA | 42 |
| | GTCTCGTGGGCTCGG-3′ | |
| ont-BC31 | 5′-GTCTCGTCCGCTCGGCAATGTAACTGATTGCTGTACGCA | 43 |
| | GTCTCGTGGGCTCGG-3′ | |
| ont-BC32 | 5′-GTTAGTTGATGTAGTATGACGTTGTCGGACTTCTACTGG | 44 |
| | GTCTCGTGGGCTCGG-3′ | |
| ont-BC33 | 5′-GTCTCGTCCGCTCGGAGTTACCCAACCGTACCAAGTCTG | 45 |
| | GTCTCGTGGGCTCGG-3′ | |
| ont-BC34 | 5′-GTTAGTTGATGTAGTGCCTTTGACTTGAGTTCTTCGTCC | 46 |
| | GTCTCGTGGGCTCGG-3′ | |
| ont-BC35 | 5′-GTCTCGTCCGCTCGGGCAGTCCCTCAGCTTCGTAAGTAG | 47 |
| | GTCTCGTGGGCTCGG-3′ | |
| ont-BC36 | 5′-GTTAGTTGATGTAGTTGTTTCCTCCTCTAACTGGGACAT | 48 |
| | GTCTCGTGGGCTCGG-3′ | |
| ont-BC37 | 5′-GTCTCGTCCGCTCGGTGATACTAAGCATCAATCGCAAGC | 49 |
| | GTCTCGTGGGCTCGG-3′ | |
| ont-BC38 | 5′-GTTAGTTGATGTAGTTTCTCTGTATCGTCCTCCTGTGGT | 50 |
| | GTCTCGTGGGCTCGG-3′ | |
| ont-BC39 | 5′-GTTAGTTGATGTAGTGAGAGGCTCTAGTTGACACTGTGG | 51 |
| | GTCTCGTGGGCTCGG-3′ | |
| ont-BC40 | 5′-GTTAGTTGATGTAGTGGCTATCCTTGGTCATCCAAACTA | 52 |
| | GTCTCGTGGGCTCGG-3′ | |
| ont-BC41 | 5′-GTTAGTTGATGTAGTCGTGTACTTCTCTGGACGAACTCC | 53 |
| | GTCTCGTGGGCTCGG-3′ | |
| ont-BC42 | 5′-GTCTCGTCCGCTCGGCTGGCAGGTATGCCTTACACGTAG | 54 |
| | GTCTCGTGGGCTCGG-3′ | |
| ont-BC43 | 5′-GTTAGTTGATGTAGTCTACCGTCGAGTCAACAACGAAAG | 55 |
| | GTCTCGTGGGCTCGG-3′ | |
| ont-BC44 | 5′-GTTAGTTGATGTAGTGAGTGGGAAGGAACCCTTTCTACT | 56 |
| | GTCTCGTGGGCTCGG-3′ | |
| ont-BC45 | 5′-GTCTCGTCCGCTCGGCACTGAAGGCATCTCTGTTGGATC | 57 |
| | GTCTCGTGGGCTCGG-3′ | |
| ont-BC46 | 5′-GTTAGTTGATGTAGTCAGGAGAATGAAGTGGAACACAGC | 58 |
| | GTCTCGTGGGCTCGG-3′ | |
| ont-BC47 | 5′-GTCTCGTCCGCTCGGGAACTACCTGTGGGAAAGTTGCAC | 59 |
| | GTCTCGTGGGCTCGG-3′ | |
| ont-BC48 | 5′-GTTAGTTGATGTAGTTACAGGTGTACCACGTTCCAGATG | 60 |
| | GTCTCGTGGGCTCGG-3′ | |
| ont-BC49 | 5′-GTCTCGTCCGCTCGGCTAGATGTTCAAAGCTGCACCAGT | 61 |
| | GTCTCGTGGGCTCGG-3′ | |
| ont-BC50 | 5′-GTTAGTTGATGTAGTACGCAGGAAGTTACCAAAGTCCAT | 62 |
| | GTCTCGTGGGCTCGG-3′ | |
| ont-BC51 | 5′-GTCTCGTCCGCTCGGGAGGACCCAGTAGGCTCATTCAAC | 63 |
| | GTCTCGTGGGCTCGG-3′ | |
| ont-BC52 | 5′-TACATTGATGCATGGGTCCACGAACAATCTTGTCTCTCA | 64 |
| | GTCTCGTGGGCTCGG-3′ | |
| ont-BC53 | 5′-GTCTCGTCCGCTCGGCTTTGCATGAGACGGTCTGAATCT | 65 |
| | GTCTCGTGGGCTCGG-3′ | |
| ont-BC54 | 5′-GTTAGTTGATGTAGTCATGCTCCTTAGTCAAAGCTCTTG | 66 |
| | GTCTCGTGGGCTCGG-3′ | |
| ont-BC55 | 5′-GTCTCGTCCGCTCGGCGTAGATCAGGGTCTCATCTTCCA | 67 |
| | GTCTCGTGGGCTCGG-3′ | |
| ont-BC56 | 5′-GTCTCGTCCGCTCGGTTCATGCCACCTGTTGAGTAGTGA | 68 |
| | GTCTCGTGGGCTCGG-3′ | |
| ont-BC57 | 5′-TACATTGATGCATGGACTTCCGAAGGAGATTGACCTAGC | 69 |
| | GTCTCGTGGGCTCGG-3′ | |
| ont-BC58 | 5′-GTTAGTTGATGTAGTTCAGACTCACGGAGGAGTAACCTG | 70 |
| | GTCTCGTGGGCTCGG-3′ | |
| ont-BC59 | 5′-GTTAGTTGATGTAGTACCTTGCTTTCCCTTCTTGATTGA | 71 |
| | GTCTCGTGGGCTCGG-3′ | |
| ont-BC60 | 5′-GTTAGTTGATGTAGTCCATAGAAGCCTTGGTTGAACATG | 72 |
| | GTCTCGTGGGCTCGG-3′ | |
| ont-BC61 | 5′-TACATTGATGCATGGGTGCTGAGGCACATAGTACCCTCT | 73 |
| | GTCTCGTGGGCTCGG-3′ | |
| ont-BC62 | 5′-GTTAGTTGATGTAGTTACGTCCTGAAGTAAGTGTGGGTG | 74 |
| | GTCTCGTGGGCTCGG-3′ | |
| ont-BC63 | 5′-GTTAGTTGATGTAGTGTTCAAGACCCAGGAACTTCAGAA | 75 |
| | GTCTCGTGGGCTCGG-3′ | |
| ont-BC64 | 5′-GTTAGTTGATGTAGTGAAAGTCGATGAACGGTGTCTGTC | 76 |
| | GTCTCGTGGGCTCGG-3′ | |
| ont-BC65 | 5′-GTCTCGTCCGCTCGGCCTTGTCTGGAGGAAGACTGAGAA | 77 |
| | GTCTCGTGGGCTCGG-3′ | |
| ont-BC66 | 5′-GTCTCGTCCGCTCGGGAAGTTAGAAGCCACAAGGATCGG | 78 |
| | GTCTCGTGGGCTCGG-3′ | |
| ont-BC67 | 5′-TACATTGATGCATGGGGTGAGCACACGAGTATGACAAAC | 79 |
| | GTCTCGTGGGCTCGG-3′ | |
| ont-BC68 | 5′-GTCTCGTCCGCTCGGCCACCTTCGTGTTTGCTTAGATTC | 80 |
| | GTCTCGTGGGCTCGG-3′ | |
| ont-BC69 | 5′-GTTAGTTGATGTAGTAGATCACATGAGGCTCGGACTGTA | 81 |
| | GTCTCGTGGGCTCGG-3′ | |
| ont-BC70 | 5′-GTTAGTTGATGTAGTACACTCCATTCGTAGGATCTCGGT | 82 |
| | GTCTCGTGGGCTCGG-3′ | |
| ont-BC71 | 5′-GTCTCGTCCGCTCGGCTGTTACTACCTGATGCTCCCAGG | 83 |
| | GTCTCGTGGGCTCGG-3′ | |
| ont-BC72 | 5′-GTTAGTTGATGTAGTGTCGGTATGGAAGACAGTCAGCTA | 84 |
| | GTCTCGTGGGCTCGG-3′ | |
| ont-BC73 | 5′-GTCTCGTCCGCTCGGGAGGGTTCTGTCATCCTGTTTCTT | 85 |
| | GTCTCGTGGGCTCGG-3′ | |
| ont-BC74 | 5′-GTTAGTTGATGTAGTAGTGGAAGTGTTGGGATGCTTGTA | 86 |
| | GTCTCGTGGGCTCGG-3′ | |
| ont-BC75 | 5′-GTCTCGTCCGCTCGGACAACAGGGTTCATCACAATGGTC | 87 |
| | GTCTCGTGGGCTCGG-3′ | |
| ont-BC76 | 5′-GTTAGTTGATGTAGTGTCCAGGGTTGATGTAACAAGCAT | 88 |
| | GTCTCGTGGGCTCGG-3′ | |
| ont-BC77 | 5′-GTCTCGTCCGCTCGGGTTGTATCCCTGAGAAACAGGTCG | 89 |
| | GTCTCGTGGGCTCGG-3′ | |
| ont-BC78 | 5′-GTTAGTTGATGTAGTTTCTGATTCAAAGGTTCGGTTGTT | 90 |
| | GTCTCGTGGGCTCGG-3′ | |
| ont-BC79 | 5′-GTCTCGTCCGCTCGGCAGCAGTGAGAACTATCTCCGAGA | 91 |
| | GTCTCGTGGGCTCGG-3′ | |
| ont-BC80 | 5′-GTTAGTTGATGTAGTGAATCGCTATCCTATGTTCATCCG | 92 |
| | GTCTCGTGGGCTCGG-3′ | |
| ont-BC81 | 5′-GTCTCGTCCGCTCGGCCGAAACAACTTCACAAGATGAGG | 93 |
| | GTCTCGTGGGCTCGG-3′ | |
| ont-BC82 | 5′-GTTAGTTGATGTAGTTAGTCCTGGAACTCGACATACCGT | 94 |
| | GTCTCGTGGGCTCGG-3′ | |
| ont-BC83 | 5′-GTCTCGTCCGCTCGGTTCGACCTTACCTAGATCAAGCCA | 95 |
| | GTCTCGTGGGCTCGG-3′ | |
| ont-BC84 | 5′-GTTAGTTGATGTAGTTGGCACAGGTTCTAGGTCCACTAC | 96 |
| | GTCTCGTGGGCTCGG-3′ | |
| ont-BC85 | 5′-GTCTCGTCCGCTCGGGATCATCCAACTAACTCCTCCGTT | 97 |
| | GTCTCGTGGGCTCGG-3′ | |
| ont-BC86 | 5′-GTCTCGTCCGCTCGGTACTTACGCTTGTTGGGATCACCT | 98 |
| | GTCTCGTGGGCTCGG-3′ | |
| ont-BC87 | 5′-GTCTCGTCCGCTCGGCCTCCCTAACAACAGGAGCATGTA | 99 |
| | GTCTCGTGGGCTCGG-3′ | |
| ont-BC88 | 5′-GTTAGTTGATGTAGTCTGCTTCGGATCGGTAGTAGAAGA | 100 |
| | GTCTCGTGGGCTCGG-3′ | |
| ont-BC89 | 5′-GTCTCGTCCGCTCGGCAACTAGCCAAACATTGATGCTGT | 101 |
| | GTCTCGTGGGCTCGG-3′ | |
| ont-BC90 | 5′-GTTAGTTGATGTAGTGCCTCAAACCGTACCCTCTACATC | 102 |
| | GTCTCGTGGGCTCGG-3′ | |
| ont-BC91 | 5′-TACATTGATGCATGGAGTAGCGTGAGTTCCTATGGAGCC | 103 |
| | GTCTCGTGGGCTCGG-3′ | |
| ont-BC92 | 5′-GTCTCGTCCGCTCGGGGTCCTGTATCTTTCCACTCACAA | 104 |
| | GTCTCGTGGGCTCGG-3′ | |
| ont-BC93 | 5′-GTCTCGTCCGCTCGGCCCAAGTCTGAAGTGATGGAAACT | 105 |
| | GTCTCGTGGGCTCGG-3′ | |
| ont-BC94 | 5′-GTTAGTTGATGTAGTGTAGGTGGCAGTTTGAGGACAATC | 106 |
| | GTCTCGTGGGCTCGG-3′ | |
| ont-BC95 | 5′-GTCTCGTCCGCTCGGAAGTCCATTCTTCTTCCAGACAGG | 107 |
| | GTCTCGTGGGCTCGG-3′ | |
| ont-BC96 | 5′-TACATTGATGCATGGATGGTGGACTCTATGACCGTTCAG | 108 |
| | GTCTCGTGGGCTCGG-3′ | |

The linker is marked with italics in the sequence in Table 3.

### Gel Electrophoresis, PCR2-Products and Clean Pooled PCR2-Product

10 μL of the PCR2-samples was added to a 2.5% agarose gel stain with MIDIGREEN, for 30 minutes at 120V, and visualized under UV Transillumination.

### Pooled PCR2-Samples

Product of PCR2 was pooled into one clean 1.5 mL Eppendorf tube and the above described clean-up protocol was carried out, though with an adjusted end volume at 50 μL instead of 15 μL. To ensure a successful clean-up, 10 μL of the pooled sample was added to a 2.5% agarose gel stained with MIDIGREEN and run for 30 minutes at 120V.

### Library Preparation, Multilevel Multiplexing

As the 4×96 samples are barcoded with barcodes according to Table 3 (sample barcode), samples are further barcoded with a native barcode using NB09, NB10, NB11 and NB12 (Table 4) (group barcode).

Library preparation was carried out following the manufacture's protocol Oxford Nanopore Technologies, Native barcoding amplicons (with EXP-NBD104, EXP-NBD114, and SQK-LSK109). For optimization, 24 μL 100-200 fmol end-prepped DNA was added and the cleaning process was carried out with 75% EtOH. There were no other further changes to the protocol.

In brief, 100-200 fmol amplicon DNA was transferred and adjusted to a volume of 48 μl in a thin-walled PCR tube for end-prep. 3.5 μl Ultra II End-prep reaction buffer and 3 μl Ultra II End-prep enzyme mix was added to the solution and mixed thoroughly by pipetting. PCR tube was spun down before incubation in a thermal cycler (20° C. for 5 minutes and 65° C. for 5 minutes).

After incubation, clean-up was carried out on the solution by AMPure XP beads. AMPure XP beads were resuspended by vortexing and placed on a Hula-Mixer. The end-prep solution was transferred to a clean 1.5 mL Eppendorf DNA LoBind tube where 60 μl of resuspended AMPure XP beads were added. The solution was mixed by flicking and placed on a Hula Mixer for further incubation for 5 minutes at room temperature. After incubation, the solution was placed on a magnet rack where the supernatant became clear. It was removed and 200 μl of 500 μl of fresh 75% Ethanol in Nuclease-free water was added without disturbing the pellet. The Ethanol was removed and discarded. This step was repeated. Any residual Ethanol was removed by spinning down the tube and place it back on magnet rack. Following, it was dried for approximately 30 seconds without drying the pellet to the point of cracking. The tube was removed from magnet rack, 25 μl of Nuclease-free water added and the pellet resuspended, spun down and incubated for 2 minutes at room temperature. The tube was placed back on the magnet rack and the clear and colourless solution was transferred to a clean 1.5 mL Eppendorf DNA LoBind tube. 1 μl of eluted sample was quantified using a Qubit fluorometer.

For ligation of native barcodes, native barcodes and Blunt/TA Ligase Master Mix were thawed in a cooling block. One barcode per sample was used (NB09, NB10, NB11 and NB12). 100-200 fmol of each end-prepped sample to 22.5 μl was diluted in Nuclease-free water. 2.5 μl Native barcode and 25 μl Blunt/TA Ligase Master Mix was added to the solution and mixed by pipetting. The mixture was incubated for 10 minutes at room temperature.

Hereafter, clean-up was carried out on the solution by AMPure XP beads. AMPure XP beads were resuspended by vortexing and placed on a Hula-Mixer. The end-prep solution was transferred to a clean 1.5 mL Eppendorf DNA LoBind tube where 60 μl of resuspended AMPure XP beads were added. The solution was mixed by flicking and placed on Hula Mixer for further incubation for 5 minutes at room temperature. After incubation, the solution was placed on a magnet rack where the supernatant beame clear. Then, it was removed and 200 μl of 500 μl of fresh 75% Ethanol in Nuclease-free water was added without disturbing the pellet. The Ethanol was removed and discarded. This step was repeated. Any residual Ethanol was removed by spinning down the tube and place it back on magnet rack. It was allowed to dry for approximately 30 seconds without drying the pellet to the point of cracking. The tube was removed from the magnet rack and 26 μl of Nuclease-free water was added and the pellet resuspended, spun down and incubated for 2 minutes at room temperature. The tube was placed back on the magnet rack and the clear and colourless solution was transferred to a clean 1.5 mL Eppendorf DNA LoBind tube. 1 μl of eluted sample was quantified using a Qubit fluorometer. Equimolar amounts of each barcoded sample were pooled into a 1.5 mL Eppendorf DNA LoBind tube, ensuring that sufficient samples was combined to produce a pooled sample of 100-200 fmol. 1 μl of eluted sample was quantified using a Qubit fluorometer.

Following a sequencing adapter was ligated to the samples as follows: Reagents (Elution Buffer, NEBNext Quick Ligation Reaction Buffer x5, T4 Ligase, Adapter Mix II, Short Fragment Buffer) were thawed in a cooling block, mixed by vortexing, spun down and placed back in a cooling block in the fridge. In the performances of the adaptor ligation the pooled and barcoded DNA (65 μl 100-200 fmol) were mixed with Adaptor Mix II (5 μl), NEBNext Quick Ligation Reaction Buffer x5 (20 μl) and Quick T4 DNA Ligase (10 μl) and set for incubation for 10 minutes at room temperature.

The reaction was hereafter cleaned with AMPure XP beads and washed with Short Fragment Buffer. After clean-up, Elution Buffer was added, the pellet resuspended and hereafter incubated for 10 minutes at room temperature. The solution was placed on a magnet rack, where the supernatant was removed and retained in a clean 1.5 mL Eppendorf DNA LoBind tube. Quantification of adapter ligated DNA was carried out using a Qubit fluorometer.

TABLE 4
Native barcoding components used

| Name | Sequence | SEQ ID NO. |
|---|---|---|
| NB09 | 5′-AACCAAGACTCGCTGTGCCTAGTT-3′ | 109 |
| NB10 | 5′-GAGAGGACAAAGGTTTCAACGCTT-3′ | 110 |
| NB11 | 5′-TCCATTCCCTCCGATAGATGAAAC-3′ | 111 |
| NB12 | 5′-TCCGATTCTGCTTCTTTCTACCTG-3′ | 112 |

The samples were primed and loaded for sequencing according to following manufacture's protocol (Oxford Nanopore Technologies, Native barcoding amplicons (with EXP-NBD104, EXP-NBD114, and SQK-LSK109)). In brief, sequencing Buffer, Loading Beads, Flush Tether and Flush Buffer were thawed at room temperature. 30 μl of Flush Tether was added to the tube of Flush Buffer and mixed by vortexing. 800 μl of the Flush mixture was added to the priming port and left for 5 minutes. Loading Beads were mixed thoroughly by pipetting. 12 μl of 50 fmol DNA library was transferred to a clean Eppendorf DNA LoBind tube along with 25.5 μl Loading Beads and 37.5 Sequencing Buffer. To complete the flow cell priming, 200 μl of the Flush mixture was added via the priming port. The prepared library was gently mixed and a 75 μl of the library solution was added via the SpotON sample port.

### Phivea® Platform

Cytogenetic chromosome analysis was carried out by Phivea® platform.

### Data Sequencing

Oxford Nanopore sequencing data is stored in two file types, .fast5 and .fastq. Base-calling summary information is stored in a sequencing_summary.txt file:
.fast5 is a customized file format based upon the .hdf5 file type, which is designed to contain all information needed for analysing nanopore sequencing data, including raw signal data, and tracking it back to its source. As default each fast5 file will contain 4000 reads although this can be configured when starting a run.
.fastq is a universal text-based sequence format for storing a biological sequence (nucleotide sequence) and its corresponding quality scores, generated when the nanopore signal data is base-called. Both the sequence letter and quality score are each encoded with a single ASCII character for brevity. By default the device saves up to 4000 sequences in one .fastq file. A .fastq file uses four lines per sequence:
   The first line begins with a ‘@’ character and is followed by a sequence identifier and an optional description.
   The second line is the raw sequence letters.
   The third line begins with a ‘+’ character and is optionally followed by the same sequence identifier (and any description) again.
   The fourth line encodes the quality values for the sequence in the second line and must contain the same number of symbols as letters in the sequence.
sequencing_summary.txt contains metadata about all base-called reads from an individual run. Information includes read id, sequence length, per-read qscore, duration etc.

The Phivea® platform uses only the .fastq files generated by the Oxford Nanopore Technologies GridION x5 device and accesses them using a shared file system. Every information related to the reads generated by the GridION x5 device is presented in real-time to the human operator using a user interface (such as the number of generated and processed .fastq files, the number of sequences, the quality of the reads and the status of the analysis). Once the .fastq files are generated and shared with the Phivea® platform that analysis process is initiated.

### Analysis Process

The analysis process includes four different processing phases:
1. Quality check
2. Demultiplexing (person classification)
3. Chromosome classification
4. Chromosomal aberration classification

In the first phase (quality check), every sequence from the .fastq file (one .fastq file contains 4000 sequences) must meet certain quality criteria. The length of the sequence must be between 900 and 1200 characters and its average Phred quality score has to be higher than 8 (88% of probability that the sequence matches the pattern). The quality score is calculated based on the average quality values of each character. If the sequence is longer than 1700 characters, we split the sequence and treat it as two separate sequences. The barcode assigned to the particular sample is used as a splitting point. If the barcode is not found in the middle of the sequence (between the 100th character from left and the 100th character from right of the middle character), then the sequence is rejected and it is not used for further processing. The barcode search is performed using a sliding window technique with size 24 and stride 1. The distance between the window and the barcode is defined using the Levenshtein distance and it should be less than 5. This phase has low computational complexity and can be performed in real-time (after the DNA reads are written in the .fastq files).

In the second phase, barcode classification or demultiplexing of the DNA sequence is performed. In particular, the barcode that encodes the information about the person to which the sequence read is associated should be identified. The proposed algorithm uses Levenshtein distance to find the location of the spacer (15 characters) and linker (15 characters) first, and then to identify the barcode (24 characters) located between the spacer and the linker. The algorithm performs the search on the first and the last 150 characters of the sequence. First, we try to find the spacer/linker by comparing a sliding window (with size 15 and stride 1) to the known values with Levenshtein Distance less than 5.

We search the spacer first, and then the linker. The barcode should follow directly after the spacer and before the linker. Once the start and the end of the barcode are identified we find the most similar barcode from the database and perform the person classification-see table here below.

TABLE
(SEQ ID NO. 113)

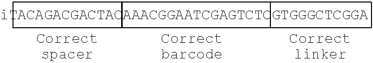

Sometimes, the spacer or the linker could be corrupted (e.g. lab preparation, sequencing error or others sources), so the start and the end of the barcode could not be identified precisely. In that case we take the 24 characters that follow the spacer and try to match with the barcodes in the database. The same procedure is repeated for the 24 characters that precede the linker. The sequence that has lower Levenshtein Distance with the barcodes in the database is used for the person classification. Again, the maximum Levenshtein Distance which is acceptable for person classification is less than 5. See also table here below:

TABLE
(SEQ ID NO. 114)

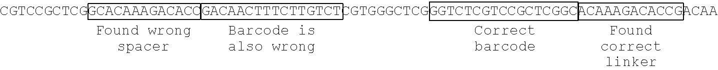

To identify the end of the human DNA sequence we use the same approach. We perform the search on the last 150 characters taking into account that the linker is in opposite order with the replacement of characters T and G with A and C due to the reverse-complement representation.

The proposed demultiplexing method managed to significantly reduce the computational complexity of the barcode identification, while preserving the quality of classification compared to the prior art competing methods. It exceeds the limits for real-time barcode identification and DNA sample analysis. We compared its computational efficiency and predictive performance with the state-of-the-art demultiplexing method guppy on a next-generation sequencing run using 6 different DNA samples. The experimental validation was performed on 1 184 898 base-called DNA reads (sequence length: 900-1200) with a Phred quality score higher than 8 as a ground truth.

In terms of computational efficiency, the proposed method demultiplexed the base-called DNA reads by an order of magnitude faster than guppy. The calculated throughput of the proposed method was ~1520 reads/s, while the calculated throughput of guppy was only ~138 reads/s. Also, it managed to significantly reduce the number of unclassified reads (6.7%) in comparison to guppy (24%). In terms of classification performance, both methods showed very similar results. The precision and the recall of the proposed method was 97.7% and 81.4% respectively, while guppy showed precision of 97.8% and recall of 81.3%. All the experiments were performed on one referent hardware architecture (Intel i7 10th generation, 8 cores, 32 GB RAM, no CUDA) using thread parallelism of 10.

In the third phase, chromosome classification is performed. For each part of the identified human DNA sequence, the BLAST algorithm is used to compare the sequence with a library and to find the sequence characteristic specific for a particular chromosome. As an output from this phase, we obtain the chromosome sequences and their number grouped by barcode and chromosome. In this experiment, 5 different chromosomes were used (represented by columns chY, chX, ch21, ch18, ch13 and ch15 in table 5). The numbers in the different columns represent the number of recognized chromosomes from the sequence reads for each individual barcode. For example, for the barcode BRK01, 8 sequence reads are recognized as chromosome Y, 40137 sequence reads are recognized as chromosome X, and so on. The total number of recognized sequence reads for BRK01 is 124271. The 8 sequence reads for Y chromosome is found to be an acceptable background in a female control sample.

TABLE 5
The number of classified reads grouped per chromosome and barcode

| | chY | chX | ch21 | ch18 | ch13 | ch15 | total |
|---|---|---|---|---|---|---|---|
| BRK01 | 8 | 40137 | 33062 | 10671 | 17908 | 22485 | 124271 |
| BRK02 | 7 | 33773 | 21460 | 14145 | 13048 | 23360 | 105793 |

In the fourth phase, genetic disorder classification is made. For this experiment, we built a machine learning binary classifier for Klinefelter syndrome classification. This phase is divided into two independent stages: offline and online as illustrated in FIG. 7.

Offline stage: In this stage, we trained a classification model and performed parameter tuning by cross validation using 278 samples (68 Klinefelter syndrome, 198 healthy, 4 trisomy syndrome, 4 AMS and 4 Prader-Willi syndrome samples), obtained from 50 different persons, generated in 5 individual runs on the Oxford Nanopore Technologies GridION x5 device. In each individual run we introduced 4 male (XY) and 4 female (XX) healthy control samples, which are used to estimate the quality of the run and the variance between the different runs.

To discriminate against Klinefelter syndrome, we built a machine learning binary classifier that uses all 278 samples for training. All Klinefelter syndrome samples (68) were labeled as positive and all the other samples (including healthy and non Klinefelter syndrome samples) as negative (210). The parameter tuning of the classifier was performed using stratified 5 fold cross validation, where 80% of the data were used for training and the other 20% of the data for classifier validation. In this experiment, SVM was used as a binary classifier.

The samples from the dataset were represented with 8 continuous variables (features). Each sample from the dataset is represented with:
1. The normalized discrete probability distribution of the occurrence of the chromosomes for that particular sample (columns p-chY, p-chX, p-ch21, p-ch18, p-ch13 and p-ch15, table 6).
2. The average Euclidean distance from the normalized discrete probability distribution of the sample to the normalized discrete probability distributions of the 4 male healthy control samples from the corresponding run (column “distance” in the table).
3. The ratio between the chY and chX probabilities.

TABLE 6
Example of the dataset (after normalization and feature engineering)

| | p-chY | p-chX | p-ch21 | p-ch18 | p-ch13 | p-ch15 | distance | chY/chX |
|---|---|---|---|---|---|---|---|---|
| BRK29 | 0.168 | 0.165 | 0.177 | 0.127 | 0.139 | 0.224 | 0.023 | 1.020 |
| BRK30 | 0.149 | 0.148 | 0.181 | 0.147 | 0.129 | 0.246 | 0.025 | 1.004 |

Online stage: In this stage, we perform the classification of new, unseen samples using the model built in the offline stage. The new, unseen samples are represented with the same features as the samples used for training:
1. The normalized discrete probability distribution of the occurrence of the chromosomes for the unseen (unlabeled) sample.
2. Average Euclidean distance from the normalized discrete probability distribution of the sample to the normalized discrete probability distributions of the 4 male healthy control samples from the new, testing run.
3. The ratio between the chY and chX probabilities for the unseen (unlabeled) sample.

Once, a new sample is generated by the processing pipeline, the classifier is consulted and the final decision about the sample is made (positive—a Klinefelter syndrome, negative-non Klinefelter syndrome or inconclusive—the probability of being Klinefelter syndrome is higher than 0.75 and the probability of being non Klinefelter syndrome is lower than 0.1).

All the phases in the analysis process are performed in real-time. This means that the filtering (quality check), demultiplexing, chromosome classification and the online stage from the genetic disorder classification are processed immediately after the DNA reads are written in the .fastq files. Their combined computational complexity is lower than the computational complexity of the DNA sequence reads generation by the ONT device. This means that the Phivea® platform can be applied in real-time on the stream of base-called DNA reads that are generated by the ONT device to monitor and analyse the health status and the condition of the whole process. It can make smart decisions related to the quantity and the quality of the reads based on some predefined criteria. This enables additional cost optimization in situations of faulty lab experiment design (using the early-stop criteria) or completed diagnostic over the full patient's list (using the criteria for sufficient number of reads processed by patient and diagnosis decision made). All of this information is presented in real-time to the human operator, so he/she is able to manage and control the whole process

### Experimental Design and Results

The evaluation of the classifier is performed using 2 positive biological samples from 2 Klinefelter syndrome patients in comparison to either negative samples from two healthy donors or 10 biological samples from patients with other chromosomal abnormalities (see Table 1). Data from the healthy controls was used to calculate the analytical and the diagnostic sensitivity and data from patients with a chromosomal abnormality different from Klinefelter syndrome were used to calculate the analytical and the diagnostic specificity. Each biological sample was evaluated in quadruplicates (one different barcode for each technical replicate) to ensure repeatability. The samples were prepared and analysed as described above.

The number of true positive (TP), true negative (TN), false positive (FP) and false negative (FN) from four different sequencing runs were calculated overall for each independent technical replicate (that is, for each barcode independently) but also, per sample (that is, for each four replicates assuming that 75% of the results were coincident, either positive or negative). Inconclusive results (either at barcode level or at patient level) were not taken into account for TP, TN, FP or FN calculations.

Klinefelter positive samples were analyzed 10 times (that is, with 40 different barcodes). Of the 40 replicates, 32 were classified by the software as “positive” for Klinefelter, that is 80% TP, 1 was classified as “negative”, that is 2.5% FN and 7 were classified as “inconclusive” (17.5%). The 32 TP allowed classifying as “positive” 7 samples (replicates of 4) and the FN did not have an impact on analytical sensitivity as the three remaining replicates for that patient in particular were classified as “positive”. Three of the 10 samples were classified as inconclusive (30%). Healthy control samples were analyzed 8 times (that is, with 32 different barcodes). Of the 32 replicates, 30 were classified by the software as “negative” for Klinefelter, that is 94% TN, and 2 were classified by the model as “inconclusive” (6%). These two inconclusive replicates belonged to the same patient/run, impeding correctly classifying this sample as negative (and leaving it as inconclusive); however, this did not have an impact on sensitivity or specificity calculations.

Samples from the other chromosomal abnormalities were analyzed 24 times (that is, with 96 different barcodes). Of the 96 replicates, 84 were classified by the software as “negative” for Klinefelter, that is 87.5% TN (that will be added to the 30 TN from healthy donors for the calculations); 1 was classified by the software as “positive”, that is 1% FP and 13 were classified by the software as “inconclusive”, that is 13.5% (all barcodes provided a results and thus all were included in the analysis). In total, there were 27 samples (either patients from other chromosomal abnormality or healthy controls) that were correctly classified as “negative” by the system, that is 84% and 5 samples that could not be classified as they were reported as “inconclusive” (16%).

Analytical sensitivity (as a measurement of precision) was assessed as the ratio of correctly positives out of the total number of positives reported=TP/(TP+FP). At barcode level this value was =32/(32+1)=97.0%. At patient level, this value was =7/(7+0)=100%.

Diagnostic sensitivity (True Positive Rate, TPR) was assessed as the number of individuals having a positive outcome of those who actually have the condition=TP/(TP+FN). At barcode level, this value was =32/(32+1)=97.0%. At patient level, this value was =7/(7+0)=100%.

Analytical specificity was the ratio of correctly negatives out of the total number of negatives reported=TN/(TN+FN). At barcode level this value was =114/(114+1)=99.1%. At patient level, this value was =27/(27+0)=100%.

Diagnostic specificity (True Negative Rate, TNR) was assessed as the number of individuals having a negative outcome of those who actually do not have the condition=TN/(TN+FP). At barcode level, this value was =114/(114+1)=99.1%. At patient level, this value was =27/(27+0)=100%.

Accuracy was calculated as the proportion of correct predictions using the following formula=(TP+TN)/(TP+TN+FP+FN) at both barcode (98.6%) and patient levels (100%).

### Items

1. A computer implemented method for identifying, if present, a preselected chromosomal aberration, features, such as patterns, in a target sequence and/or mutated target sequence in a DNA sample obtained from a subject, the method is based on a plurality of DNA samples each originating from different subjects and/or a plurality of DNA samples representing replicas for a single subject, the method comprises
   providing a DNA sample from multiple subjects and/or from DNA samples representing replicas from a single subject;
   tagging each target sequence with a molecular barcode, wherein the tagging is a multilevel multiplexing tagging in which the plurality of target sequence are grouped into a number of groups each having a unique group barcode and each sample within a group is having a unique sample barcode within said group;
   after said tagging, sequencing said tagged target sequences in parallel by use of a sequencing platform to provide nucleotide sequences;
   de-multiplexing the nucleotide sequences on the basis of said barcodes and comparing by use of a computer nucleotide sequences, represented in a data record, with a reference nucleotide sequence, represented in a data record, and if the comparison shows a match between said target sequence of the nucleotide sequence and said reference nucleotide sequence, recording the group barcode and the sample barcode for the matched de-multiplexed nucleotide sequence.
2. A computer implemented method according to item 1, wherein the de-multiplexing and the comparing of each of the nucleotide sequences are performed in real-time and/or latency bounded by which the de-multiplexing and the comparing are carried out when nucleotides are provided by the sequencing platform.
3. A computer implemented method according to any of the preceding items, wherein sequencing platform is configured to provide sequencing results concurrently with the sequencing of one of said DNA sequence, and wherein the method is configured to abort further sequencing of a DNA sequence if said comparison shows said match.
4. A computer implement method according to any of the preceding items, wherein the multilevel multiplexing further comprises organizing said target sequences within one of said groups into at least two organizations each having a unique organization barcode within the group and wherein the tagging further includes tagging said target sequences with the organization barcodes.
5. A computer implemented method according to any of the preceding items, further comprising storing in a barcode database, the barcodes used in said tagging each being tagged with information linking a specific barcodes to a specific individual.
6. A computer implemented method according to item 5, wherein the de-multiplexing further comprising performing a database look-up in the barcode database to retrieve said information linking a barcode to a specific individual.
7. A computer implemented method according to any of the preceding items, wherein the sequencing platform is configured to parallel sequencing using single cell sequencing, such as a next generation sequencing platform, such as a nanopore DNA sequencing.
8. A computer implemented method according to any of the preceding items, wherein said molecular barcode further comprises a molecular spacer and a molecular linker, preferably said sample, group and/or organization barcode is arranged in-between said molecular spacer and said molecular linker; preferably said molecular spacer and molecular linker each comprises a known sequence of nucleotides, and wherein the de-multiplexing comprising for a DNA sequence to be de-multiplexed
   searching from the start and/or the end of said nucleotide sequence to identify said spacer and said linker by use of a sliding window, said searching comprises comparing a sequence of nucleotides of said nucleotide sequence to be de-multiplexed located within the sliding window with said known sequences of nucleotides and if a match is found for both said molecular linker and said molecular spacer assign a nucleotide sequence in between the said molecular spacer and said molecular linker to be a barcode.
9. A computer implemented method according to item 8, wherein the comparison includes evaluating a similarity measure such as a Levenshtein Distance within said window, and wherein a match is considered to be present if the similarity measure is less than a predefined threshold, such as Levenshtein Distance is less than 10, such as less than 7, such as less than 5.
10. A computer implemented method according to any of the preceding items, wherein comparing nucleotide sequences with a reference nucleotide sequence is carried out by use of a basic local alignment search tool, such as BLAST, such as VSEARCH, such as USEARH, such as UCLUST.
11. A computer implemented method according to any of the preceding items, wherein each of
   said group barcodes is a sequence of nucleotides with a length of around 6-60 nucleotides, such as 6-55 nucleotides, like 6-50 nucleotides, such as 10-40 nucleotides, like 20-30 nucleotides. In a further embodiment, the group barcode is around 6-60 nucleotides, such as 6-55 nucleotides, like 6-50 nucleotides, such as 10-40 nucleotides, like 20-30 nucleotides;
   said sample barcodes is a sequence of nucleotides with a length of around 6-60 nucleotides, such as 6-55 nucleotides, like 6-50 nucleotides, such as 10-40 nucleotides, like 20-30 nucleotides. In a further embodiment, the group barcode is around 6-60 nucleotides, such as 6-55 nucleotides, like 6-50 nucleotides, such as 10-40 nucleotides, like 20-30 nucleotides nucleotides; and
   when depending on item 3, said organization barcode is a sequence of nucleotides with a length of around 6-60 nucleotides, such as 6-55 nucleotides, like 6-50 nucleotides, such as 10-40 nucleotides, like 20-30 nucleotides. In a further embodiment, the group barcode is around 6-60 nucleotides, such as 6-55 nucleotides, like 6-50 nucleotides, such as 10-40 nucleotides, like 20-30 nucleotides.
12. A computer implemented method according to any one of the preceding items, wherein the subject is selected from the group consisting of humans, mammals, cattle, pigs, horses, sheep, goats, mink, ferrets, hamsters, birds, cats and dogs.
13. A computer implemented method according to any one of the preceding items, wherein said sequencing is carried out by use of a next generation sequencing platform, such as a third generation sequencing platform to provide nucleotide sequences.
14. A computer implemented method according to any one of the preceding items, wherein the target sequences are amplified by a PCR prior to being tagged with a sample barcode.
15. A computer implemented method according to item 14, wherein the amplified target sequences tagged with a sample barcode are amplified by a PCR prior to being tagged with a group barcode.

1: A computer implemented method for identifying, if present, a preselected chromosomal aberration, features, in a target sequence and/or mutated target sequence in a DNA sample obtained from a subject, the method is based on a plurality of DNA samples each originating from different subjects and/or a plurality of DNA samples representing replicas for a single subject, the method comprises
providing a DNA sample from multiple subjects and/or from DNA samples representing replicas from a single subject;
amplifying one more target sequences in said DNA samples, thereby providing amplified DNA samples;
tagging each target sequence with a molecular barcode, wherein the tagging is a multilevel multiplexing tagging in which:
the amplified DNA samples are grouped into a number of groups each having a unique group barcode; and
each sample and its one more target sequences within a group is having the same unique sample barcode within said group;
after said tagging, sequencing said tagged target sequences in parallel using single cell sequencing;
de-multiplexing in real-time and/or latency bounded by which the de-multiplexing and the comparing are carried out when nucleotides are provided by the sequencing platform the nucleotide sequences on the basis of said barcodes and comparing by use of a computer nucleotide sequences, represented in a data record, with a reference nucleotide sequence, represented in a data record, and if the comparison shows a match between said target sequence of the nucleotide sequence and said reference nucleotide sequence, recording the group barcode and the sample barcode for the matched de-multiplexed nucleotide sequence.

2: A computer implemented method according to claim 1, wherein the sequencing platform is configured to provide sequencing results concurrently with the sequencing of one of said DNA sequence, and wherein the method is configured to abort further sequencing of a DNA sequence if said comparison shows said match.

3: A computer implement method according to claim 1, wherein the multilevel multiplexing further comprises organizing said target sequences within one of said groups into at least two organizations each having a unique organization barcode within the group and wherein the tagging further includes tagging said target sequences with the organization barcodes.

4: A computer implemented method according to claim 1, further comprising storing in a barcode database, the barcodes used in said tagging each being tagged with information linking a specific barcodes to a specific individual.

5: A computer implemented method according to claim 4, wherein the de-multiplexing further comprising performing a database look-up in the barcode database to retrieve said information linking a barcode to a specific individual.

6: A computer implemented method according to claim 1, wherein said molecular barcode further comprises a molecular spacer and a molecular linker, preferably said sample, group and/or organization barcode is arranged in-between said molecular spacer and said molecular linker; preferably said molecular spacer and molecular linker each comprises a known sequence of nucleotides, and wherein the de-multiplexing comprising for a DNA sequence to be de-multiplexed
searching from the start and/or the end of said nucleotide sequence to identify said spacer and said linker by use of a sliding window, said searching comprises comparing a sequence of nucleotides of said nucleotide sequence to be de-multiplexed located within the sliding window with said known sequences of nucleotides and if a match is found for both said molecular linker and said molecular spacer assign a nucleotide sequence in between the said molecular spacer and said molecular linker to be a barcode.

7: A computer implemented method according to claim 6, wherein the comparison includes evaluating a similarity measure, and wherein a match is considered to be present if the similarity measure is less than a predefined threshold.

8: A computer implemented method according to claim 1, wherein comparing nucleotide sequences with a reference nucleotide sequence is carried out by use of a basic local alignment search tool.

9: A computer implemented method according to claim 3, wherein each of
said group barcodes is a sequence of nucleotides with a length of around 6-60 nucleotides;
said sample barcodes is a sequence of nucleotides with a length of around 6-60 nucleotides; and
said organization barcode is a sequence of nucleotides with a length of around 6-60 nucleotides.

10: A computer implemented method according to claim 1, wherein the subject is selected from the group consisting of humans, mammals, cattle, pigs, horses, sheep, goats, mink, ferrets, hamsters, birds, cats and dogs.

11: A computer implemented method according to claim 1, wherein said sequencing is carried out by use of a next generation sequencing platform.

12: A computer implemented method according to claim 1, wherein the target sequences are amplified by a PCR prior to being tagged with a sample barcode.

13: A computer implemented method according to claim 12, wherein the amplified target sequences tagged with a sample barcode are amplified by a PCR prior to being tagged with a group barcode.

14: A computer implemented method according to claim 1, wherein each of
said group barcodes is a sequence of nucleotides with a length of around 6-60 nucleotides; and
said sample barcodes is a sequence of nucleotides with a length of around 6-60 nucleotides.
